(19) United States
(12) Patent Application Publication
(10) Pub. No.: US 2024/0369511 A1
(43) Pub. Date: Nov. 7, 2024

(54) INTEGRATED ELECTROCHEMICAL APTASENSORS FOR MEASURING CELL DEATH AND CYTOKINE ACTIVITY FROM INTACT TISSUE SAMPLES

(71) Applicants: University of Washington, Seattle, WA (US); John Hopkins University, Baltimore, MD (US)

(72) Inventors: Albert Folch, Seattle, WA (US); Tran Nguyen, Seattle, WA (US); Lisa Horowitz, Seattle, WA (US); Netzahualcoyotl Arroyo, Baltimore, MD (US)

(73) Assignees: University of Washington, Seattle, WA (US); John Hopkins University, Baltimore, MD (US)

(21) Appl. No.: 18/652,334

(22) Filed: May 1, 2024

Related U.S. Application Data
(60) Provisional application No. 63/499,780, filed on May 3, 2023.

(51) Int. Cl. G01N 27/327; C12N 15/113; G01N 33/50

(52) U.S. Cl. CPC G01N 27/3277; C12N 15/113; G01N 33/5082; C12N 2310/16; G01N 2333/80

(57) ABSTRACT

Devices, systems, methods, and kits configured to perform bioassays on live, intact tissue for precision bioanalysis in vitro are described. These approaches enable precision oncology by capturing determinants of therapeutic response to functional drug testing, such as viability and molecular signal generation, that can depend on tissue architecture, tumor heterogeneity, and the tumor microenvironment. The disclosure provides electrochemical aptamer sensors integrated into arrays of microfluidic traps for the analysis of micro-dissected tumors, as an example. The sensors are utilized with an example of periodic monitoring of cytochrome c (Cyt-C), a soluble cell death indicator, released by micro-dissected tumors.

### CROSS-REFERENCE TO RELATED APPLICATION

This application claims the benefit of U.S. Provisional Patent Application No. 63/499,780 filed on May 3, 2023, which is incorporated by reference herein in its entirety for all purposes.

### STATEMENT OF GOVERNMENT LICENSE RIGHTS

This invention was made with government support under Grant Nos. R01CA181445 and R01CA272677, awarded by the National Cancer Institute (NCI) [NIH]. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1303US.UW_Sequence_Listing.xml. The XML file is 3,152 bytes; was created on Apr. 24, 2024; and is being submitted electronically via Patent Center with the filing of the specification.

### BACKGROUND

Cancer is a major global health issue, and advancement in effective cancer drug treatment is an ongoing challenge. A reason drugs fail in clinical trials is the poor predictive power of existing preclinical models, which is due in part to the timeline with which tissues respond to experimental drug treatments. Longer tissue response timelines typically need more samples to be taken, which is laborious because most prior efforts have related to monitoring in vivo or drug testing in vitro, and have relied on assays for measuring tissue response to drugs that have typically utilized fluorescent labeling. Fluorescent labeling is a semi-quantitative method which may be suited in some instances for single-time-point terminal assay. However, labor-intensive terminal immunostaining analysis and fluorescent labeling approaches are not as useful for continuous monitoring of intact tissues in vitro, on a long-term basis, for example, as part of monitoring responses to treatments.

Accordingly, there is a need for improved devices, systems, kits, and methods for quantifying biological agents in intact tissue samples in vitro, for example, as a result of a treatment, on a continuous or long-term basis across multiple days of measurement, rather than mere hours. The present disclosure addresses this and other long-felt and unmet needs in the art.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the disclosure provides an integrated electrochemical aptamer-based (E-AB) sensor configured to measure a concentration of an analyte, the integrated E-AB sensor comprising: an aptamer operably connected to a conductive substrate; circuitry configured to conduct square wave voltammetry (SWV) to apply an electric current to the conductive substrate; and circuitry configured to conduct kinetic differential measurement (KDM) to determine concentration of the analyte based on changes in the electric current caused by conformational changes in the aptamer upon an interaction between the aptamer and the analyte.

In embodiments of an integrated E-AB sensor, the aptamer comprises: a redox reporter configured to modify an electric current to and/or from the conductive substrate of the integrated E-AB sensor; and a nucleic acid aptamer comprising a polynucleotide sequence configured to interact with the analyte, wherein an interaction between the polynucleotide sequence and the analyte causes a conformational change in the nucleic acid aptamer, a positional change in the redox reporter, and a change in the electric current of the integrated E-AB sensor; wherein the electric current is applied with circuitry configured to conduct SWV and the change in the electric current is processed with circuitry configured to conduct KDM to determine concentration of the analyte.

In embodiments of an integrated E-AB sensor, a form factor of the integrated E-AB sensor comprises a dip-stick form factor.

In embodiments of an integrated E-AB sensor, the nucleic acid aptamer comprises deoxyribonucleic acid (DNA) and the polynucleotide sequence comprises a DNA sequence.

In embodiments of an integrated E-AB sensor, the redox reporter comprises methylene blue (MB).

In embodiments of an integrated E-AB sensor, the redox reporter is linked to the nucleic acid aptamer at a terminus of the nucleic acid aptamer.

In embodiments of an integrated E-AB sensor, the terminus with the redox reporter linked thereto is the 3′ terminus of the nucleic acid aptamer.

In embodiments of an integrated E-AB sensor, the analyte comprises Cytochrome C (Cyt-C) and the polynucleotide sequence comprises a DNA sequence specific for interaction with Cyt-C.

In embodiments of an integrated E-AB sensor, the analyte comprises Cyt-C and the polynucleotide sequence comprises a DNA sequence specific for interaction with Cyt-C, wherein the DNA sequence specific for interaction with Cyt-C comprises a DNA sequence that is at least 80% identical to the DNA sequence/5′-Thio-C6-Disulfide/CC GTG TCT GGG GCC GAC CGG CGC ATT GGG TAC GTT GTT GC/—NH₂-3′ (SEQ ID NO:1).

In embodiments of an integrated E-AB sensor, the analyte comprises Cyt-C and the polynucleotide sequence comprises a DNA sequence specific for interaction with Cyt-C, wherein the DNA sequence specific for interaction with Cyt-C is the DNA sequence/5′-Thio-C6-Disulfide/CC GTG TCT GGG GCC GAC CGG CGC ATT GGG TAC GTT GTT GC/—NH₂-3′ (SEQ ID NO:1).

In embodiments of an integrated E-AB sensor, the circuitry configured to conduct SWV uses a combined square wave applied to the conductive substrate of the integrated E-AB sensor.

In embodiments of an integrated E-AB sensor, the circuitry configured to conduct KDM to determine concentration of the analyte is configured to calculate concentration of the analyte according to:

$$[ Analyte ] = \frac{K D {M}_{min}}{{( \frac{K D {M}_{max} - K D {M}_{min}}{K D M - K D {M}_{min}} - 1 )}^{n H}}$$

wherein:
[Analyte] is the concentration of the analyte, optionally in ng/mL;
KDMₘᵢₙ is KDM observed in absence of the analyte;
KDMₘₐₓ is KDM expected at saturation of the analyte; and
nH is Hill coefficient.

In embodiments of an integrated E-AB sensor, the circuitry configured to conduct SWV and the circuitry configured to conduct KDM are implemented as a non-transitory computer-readable storage medium having instructions stored thereon which, when executed by the processor, configure the processor to: apply the electric current according to SWV; detect the change in the electric current; and process the change in the electric current according to KDM.

In an aspect, the disclosure provides a method for measuring an analyte with an integrated E-AB sensor, the method comprising: contacting a conductive substrate of the integrated E-AB sensor with a sample comprising an analyte; applying, with circuitry configured to conduct SWV, electric current to the conductive substrate; and processing, with circuitry configured to conduct KDM, a change in an electric current associated with an interaction between an aptamer of the integrated E-AB sensor and the analyte.

In embodiments of a method, the integrated E-AB sensor comprises: the aptamer operably connected to the conductive substrate; circuitry configured to conduct SWV to apply the electric current to the conductive substrate; and circuitry configured to conduct KDM to determine concentration of the analyte based on changes in the electric current caused by conformational changes in the aptamer upon an interaction between the aptamer and the analyte.

In embodiments of a method, the sample comprises a tissue sample within a solution and the analyte is from, or is produced by, the tissue sample.

In embodiments of a method, the tissue sample is selected from the group consisting of: a cuboid tissue sample, a tissue slice, an organoid tissue, and any combination thereof.

In embodiments of a method, the analyte corresponds with a response of a cuboid tissue sample of the sample to a treatment.

In an aspect, the disclosure provides a kit for measurement of an analyte, the kit comprising: an integrated E-AB sensor, comprising: an aptamer operably connected to a conductive substrate of the E-AB sensor; circuitry configured to conduct SWV to apply an electric current to the conductive substrate; and circuitry configured to conduct KDM to determine concentration of the analyte based on a change in an electric current caused by a conformational change in the aptamer upon an interaction between the aptamer and the analyte; and instructions for a use of the kit in a method for measuring the analyte with the kit.

In embodiments of a kit, the integrated E-AB sensor comprises: the aptamer operably connected to the conductive substrate; circuitry configured to conduct SWV to apply the electric current to the conductive substrate; and circuitry configured to conduct KDM to determine concentration of the analyte based on changes in the electric current caused by conformational changes in the aptamer upon an interaction between the aptamer and the analyte.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIGS. 1A-1D show an overview of an example electrochemical aptamer-based CytC monitor using microdissected tumor biopsies, according to aspects of the disclosure. FIG. 1A shows schematic illustrations of anti-cancer drug screening models. These approaches predominantly rely on endpoint analysis and do not provide real-time assessments of microdissected tissue responses to drug treatments. Integrating biosensors into the tumor-on-a-chip platforms, as disclosed herein, enables real-time, on-chip monitoring of the dynamic responses of the tissue of interest to conditions, such as contact with one or more pharmaceutical compounds. FIG. 1B shows an example layer-by-layer design of a sensor platform of the disclosure. Top to bottom: The wells layer, loading frame, and insulation layer define the sensing area, a sensor layer, and a structural support layer. The sensor layer is fabricated using conventional lithography techniques on PMMA. FIG. 1C shows an example schematic illustration of CytC sensing by an electrochemical aptamer-based sensor with the reference electrode (RE), counter electrode (CE), and working electrode (WE) functionalized with CytC aptamers. CytC-induced conformation change of the aptamers causes current response for cell-death detection. FIG. 1D shows a photograph of the example CytC sensor platform. Top left: A photo of the sensor layer on PMMA. Top right: A photo of an individual three-electrode electrochemical sensor with WE, RE, and CE. Bottom: A photo of an assembled CytC sensor platform (Scale bar: 1 cm).

FIGS. 2A-2F show characterization of the example electrochemical aptamer-based CytC sensor, according to aspects of the disclosure. FIG. 2A shows an example aptamer working mechanism. The example aptasensor comprises a redox reporter, methylene blue, and hexanethiol-modified aptamer bound to bare Au or AuNPs. In the presence of a target, e.g., Cytochrome C (CytC), aptasensors undergo a binding-induced conformational change, resulting in an electron transfer (eT) difference between the reporter and the electrode. This change can be quantified using square wave voltammetry (SWV). FIG. 2B shows an example square wave voltammogram of the CytC aptasensor. These curves represent the currents obtained during SWV scans conducted from −0.5 V to 0 V, utilizing a frequency of 50 Hz and an amplitude of 25 mV. A concentration range of CytC from 0 to 30,000 ng/mL was used (arrows indicate an increase in concentration). FIG. 2C shows a corresponding dose-response curve of CytC. Error bars indicate the standard deviation (n=4). FIG. 2D shows an example temporal response to CytC from the aptasensor. Following the addition of 1,000 ng/ml, the CytC aptasensor responds to its target within 100 s (at a SWV of 50 Hz). The shade indicates the standard deviation (n=3). FIG. 2E shows example signal responses to CytC injection over time. Initially, the aptamer remained unbound when there was no target (baseline level). Subsequently, as the target is introduced, the association phase begins, and the aptamer binding sites gradually become occupied until they reach a steady-state or equilibrium phase. Continuous buffer flow was then employed to facilitate target removal, inducing the dissociation of the aptamer-target complex and returning the signal to baseline levels. Error bars indicate the standard deviation derived from ten scans for each time point. The experiment is duplicated to confirm the results. FIG. 2F shows selectivity of CytC aptasensors with response to 100 ng/mL of CytC vs. response in the presence of relevant interferents in medium. Human fibroblast growth factor (FGF), epidermal growth factor (EGF), Interleukin-2 (IL-2), and bovine serum albumin (BSA). Error bars indicate the standard deviation (n=5).

FIGS. 3A-3D show an example Kinetic Differential Measurement (KDM) technique and characterization of Au-aptasensor, according to aspects of the disclosure. FIG. 3A shows model curves illustrating KDM. The signal gain represents the signal generated when the target concentration increases. Depending on the frequency of square-wave voltammetry used and the sensor's electron transfer rate, aptasensors can exhibit a decrease in signal gain (Signal-OFF behavior, top left figure) or an increase in signal gain (Signal-ON behavior, bottom left figure) in response to target binding. These characteristics of the aptasensor allow to calculate KDM values to enhance gain and to correct baseline drift. KDM is the difference between the normalized peak currents at Signal-ON and Signal-OFF frequency, divided by the average of Signal-ON and Signal-OFF currents. These KDM values are fitted to the Hill-Langmuir equation to create a dose-response curve (right-side graph). FIG. 3B shows a frequency map corresponding to different interrogation frequencies measured for CytC binding by Au-aptasensors, which shows dependence on frequency and regions of Signal-On and Signal-OFF frequencies. Error bars represent the standard deviation (n=5). FIG. 3C shows an example KDM measurement for CytC in DMEM-F12-10% FBS culture medium. KDM (black), as calculated from responses to CytC measured at 50 Hz and 1,000 Hz (as indicated), showed reduced drift over time and increased signal amplitude in a culture medium. Error bars represent the standard deviation (n=3 sensors). FIG. 3D shows expanded graphs of the responses to CytC for one sensor at the 360-minute mark, measured at 50 Hz then 1,000 Hz, and the calculated KDM signal. FIG. 3E shows responses of CytC aptasensors calibrated in a culture medium. Response of Au-aptasensors to CytC square wave voltammograms obtained for the 1-12,000 ng/mL concentration range at 50 Hz and 1,000 Hz (representative sensor, the arrow indicates an increase in concentration). Graph of KDM values calculated from the normalized peak height at 50 Hz and 1,000 Hz. Error bars represent the standard deviation (n=12). FIGS. 3F and 3G show comparison of actual and estimated concentration from individual sensors using dose-response curve calibration based on KDM values. FIG. 3F shows estimated concentrations were calculated from individual calibration for each sensor, and estimated concentrations were calculated from average calibration derived from all six sensors. FIG. 3G shows the percentage accuracy of estimated concentration compared to actual concentration from individual calibration for each sensor and from average calibration derived from all six sensors.

FIGS. 4A-4D show detection of CytC in the supernatant of treated human cancer cuboids using an example multiplexing system, according to aspects of the disclosure. FIG. 4A shows a photograph of the multiplexing system with PCBs for sensor interfacing and multiplexing chip housing. FIG. 4B shows a hardware block diagram for the multiplexing platform from the sensors through the multiplexers to the potentiostat. WE1: working electrode 1, RE1: reference electrode 1, CE1: counter electrode 1; MUX: multiplexer. Scale bar, 5 cm. FIG. 4C shows a schematic illustration of the supernatant CytC sensing experiment, where cuboids collected from a patient's tumor were cultured in a 24-well plate for 3 days under different drug treatment conditions, with each condition duplicated. CytC from the supernatants was collected and measured using ELISA and an electrochemical aptamer-based sensor platform. FIG. 4D shows ELISA and electrochemical sensors measurements of CytC from human DT tissues from two supernatants after treatment for three days with Staurosporine, FOLFOX, and FOLFIRI, compared to DMSO control vehicle. Individual data points represent each sensor, with lines corresponding to duplicate supernatant samples.

FIGS. 5A-5K show direct, on-device measurement of CytC secretion from microdissected tumor “cuboids” cultured and exposed to drugs on the electrochemical aptamer-based sensor platform over two days, according to aspects of the disclosure. FIG. 5A shows a schematic illustration and FIG. 5B shows photographs of an ex vivo experiment, where microdissected U87 glioma xenograft cuboids in Cellink bioink were transferred on the aptasensor system for CytC detection. FIG. 5C shows measured CytC concentrations from U87 cuboids after treatment for 48 hrs with different concentrations of cisplatin compared to control. Individual points for every sensor. n=4. FIG. 5D shows CytC corresponding concentrations in U87 cuboids after treatment for 48 hrs with YM-155 compared to DMSO control vehicle. Individual points for every sensor. n=3-5. FIG. 5E shows CytC corresponding concentrations in U87 cuboids after treatment for 48 hrs with MOC compared to DMSO control vehicle. Individual points for every sensor. n=5-6. (FIGS. 5D-5E show paired t test versus control. ns p>0.05, *p<0.05). FIGS. 5F-5H show quantitation of cell death by SYTOX Green (death) fluorescence. Mean fluorescence was normalized to the average value of control conditions. Individual points and average±S.E.M. n=25-30. (One-way ANOVA versus control, Dunnett's multiple comparison test. ns p>0.05, *p<0.05, **<0.01, **** p<0.001). FIG. 5I shows cell death in U87 cuboids after treatment for 48 hrs with different concentrations of cisplatin compared to control. FIG. 5J shows cell death in U87 cuboids after treatment for 48 hrs with YM-155 compared to DMSO control vehicle. FIG. 5K shows cell death in U87 cuboids after treatment for 48 hrs with MOC compared to DMSO control vehicle. SYTOX Green (nuclear death stain), BF (bright field). Representative images from one sensor well with four to five cuboids in each well (Scale bar: 400 μm).

FIG. 6 shows an example dry film resist fabrication process for microelectrodes, according to aspects of the disclosure. Dry film resist (DFR) is laminated and utilized as a sacrificial layer for Au deposition.

FIGS. 7A-7B show an example specification of the multiplexer chips ADG732 datasheet, according to aspects of the disclosure.

FIG. 8 shows an illustration of integrated electrochemical sensors for continuous cuboid tissue sample monitoring, according to aspects of the disclosure. The aptasensors are immobilized on gold (Au) or Au nanoparticles (AuNP). The E-AB sensors can be used for monitoring cuboid tissues, tissue slices, or organoids, as well as other forms or shapes of tissue, according to embodiments.

FIG. 9 shows a schematic and graphs demonstrating an aptasensor mechanism, according to aspects of the disclosure. KDM exploits the square wave frequency dependence of electrochemical aptamer-based (E-AB) signaling. There is a difference in electric current from the folded, target-bound aptamer and the unfolded, target-free aptamer. A binding-induced increase in current occurs when square-wave voltammetry is performed at high frequencies and a binding-induced decrease in current occurs when square-wave voltammetry is performed at low frequencies.

FIG. 10 shows detection of a cell death marker, CytC, by an aptasensor, according to aspects of the disclosure.

FIG. 11 shows continuous electrochemical monitoring of CytC from cuboids exposed to Cisplatin and Staurosporine, according to aspects of the disclosure.

FIGS. 12A-12C show an example form for an aptamer sensor, as a “dip-stick” form factor, with each sensor arranged in a tooth layout and being insertable into a well of a microwell plate for measurement of analyte, according to aspects of the disclosure. WE=working electrode (contains aptamer); CE=counter electrode; RE=reference electrode. The “dip-stick” form factor of the system is fabricated not based on plastic, but with PCB which enables laser cutting of electrodes and to have the electrodes contact the sample from above, rather than to have the sample contact the electrodes from above. This form factor may be easier to scale with a larger number of electrodes; more working electrodes provides for more analysis and/or more analysis per cell of the microfluidic platform.

### DETAILED DESCRIPTION

The disclosure provides electrochemical aptamer-based (E-AB) sensors configured for measurement of concentrations of analytes within or originated from biological substances such as living tissues, living cells, micro-dissected living tissues whether healthy or diseased (e.g., cancer tissues), in an in vitro (ex vivo) manner. The disclosed sensors integrate innovative approaches for application of electric current to the sensors, including square wave voltammetry (SWV), with kinetic differential measurement (KDM) for a significant and synergistic improvement in analyte monitoring with the platform. The disclosed sensors are capable of monitoring changes to biological substances over longer time periods, on the order of hours, days, weeks, or months, in at least some instances with decreased manual input, and enable continuous or regular monitoring for improved insight into tissue responses to established and experimental pharmaceutical treatments or culture conditions, for example.

The disclosed sensors can be put to use in any of a variety of applications, including but not limited to pre-clinical and clinical trial research and development, contract research services, measurements that pertain to immunotherapies, measurements of infectious models for monitoring changes to the immune system in response to experimental or established immunotherapy conditions or other immunological treatments, and the like.

### Electrochemical Aptamer-Based (E-AB) Sensor Devices and Systems

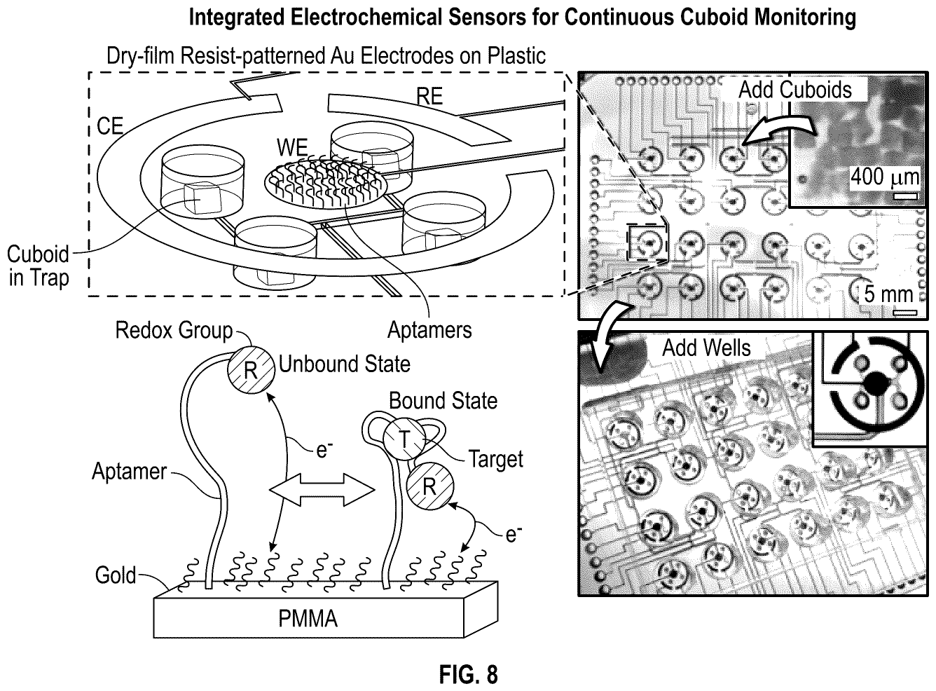

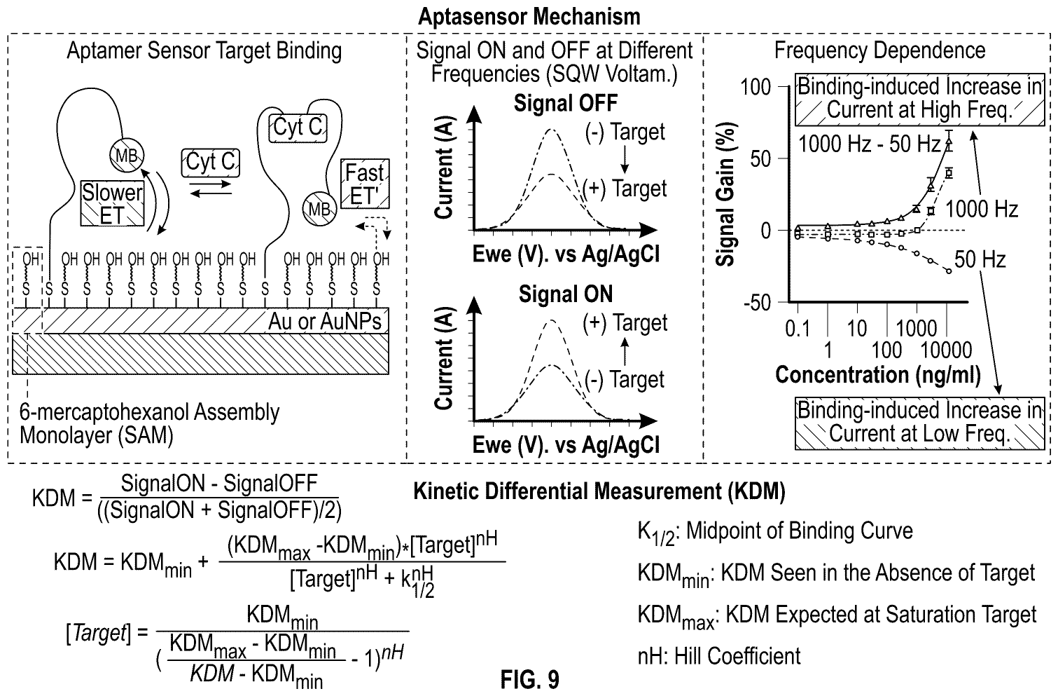

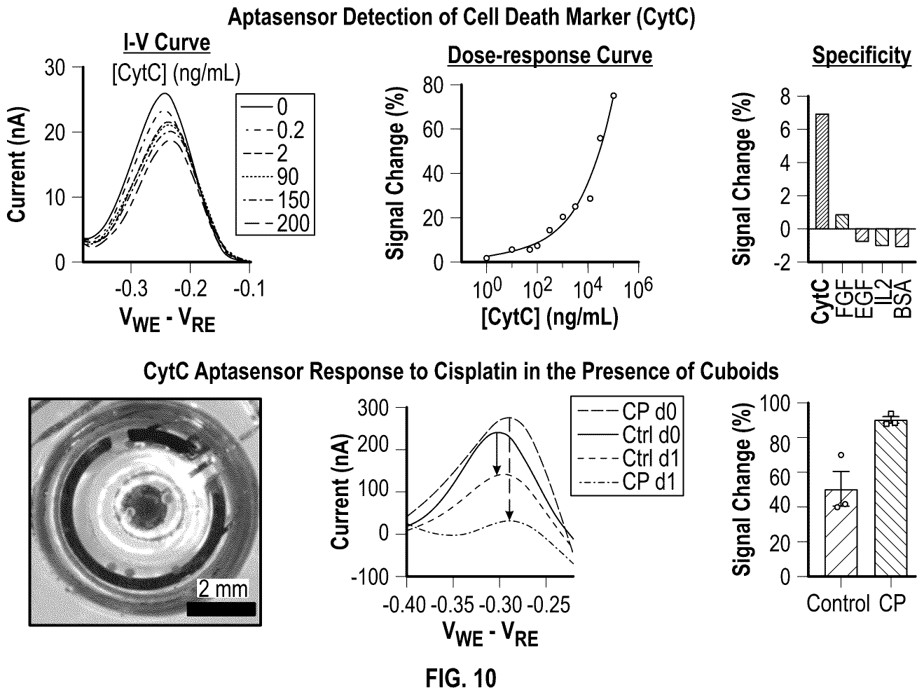

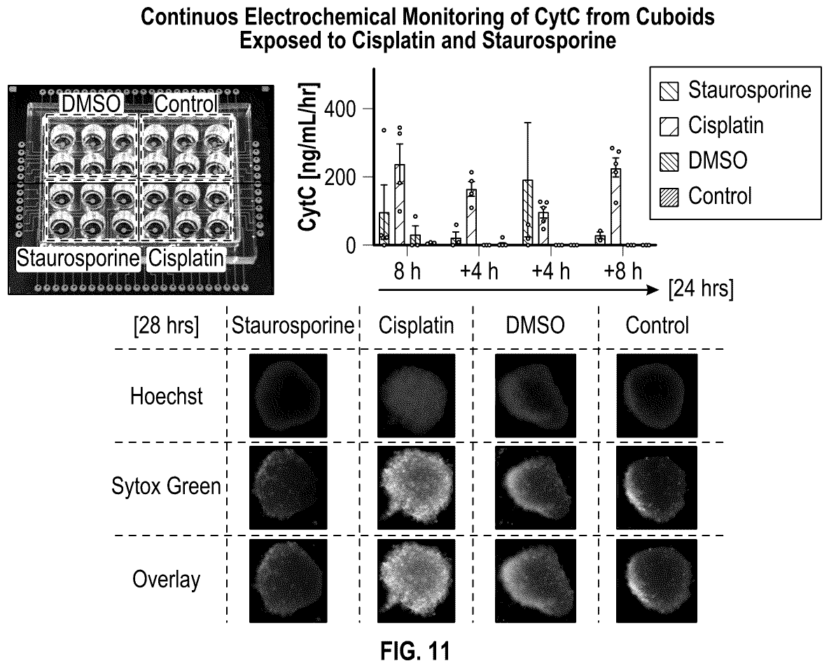

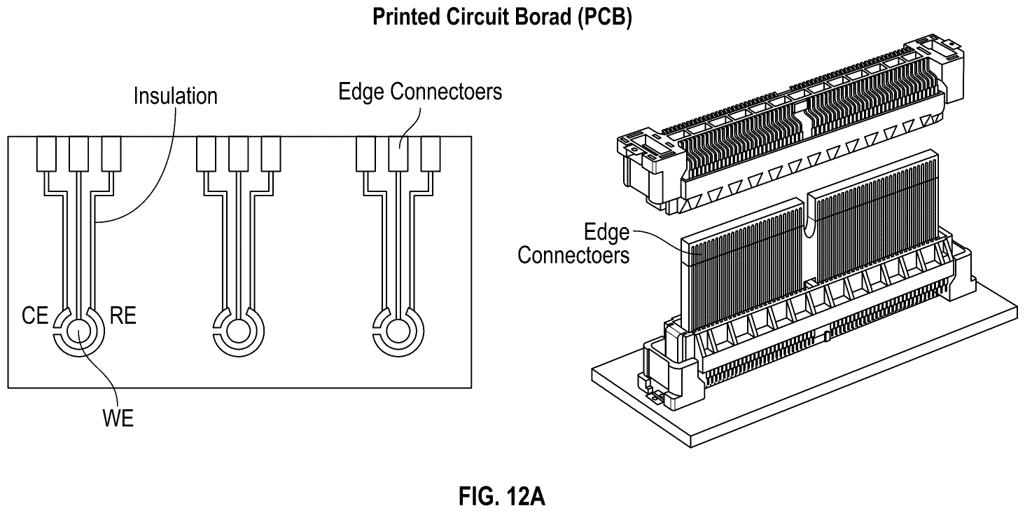

Features of an example integrated electrochemical aptamer-based (E-AB) sensor that is configured to measure a concentration of an analyte are shown at FIGS. 8, 9, 12A, 12B, and 12C. An integrated E-AB sensor comprises an aptamer operably connected to a conductive substrate, circuitry configured to conduct square wave voltammetry (SWV) to apply an electric current to the conductive substrate, and circuitry configured to conduct kinetic differential measurement (KDM) to determine concentration of the analyte based on changes in the electric current caused by conformational changes in the aptamer upon an interaction between the aptamer and the analyte. The aptamer can comprise a redox reporter configured to modify an electric current to and/or from the conductive substrate of the integrated E-AB sensor, and a nucleic acid aptamer comprising a polynucleotide sequence configured to interact with the analyte, wherein an interaction between the polynucleotide sequence and the analyte causes a conformational change in the nucleic acid aptamer, a positional change in the redox reporter, and a change in the electric current of the integrated E-AB sensor. The electric current can be applied with circuitry configured to conduct SWV and the change in the electric current can be processed with circuitry configured to conduct KDM to determine concentration of the analyte.

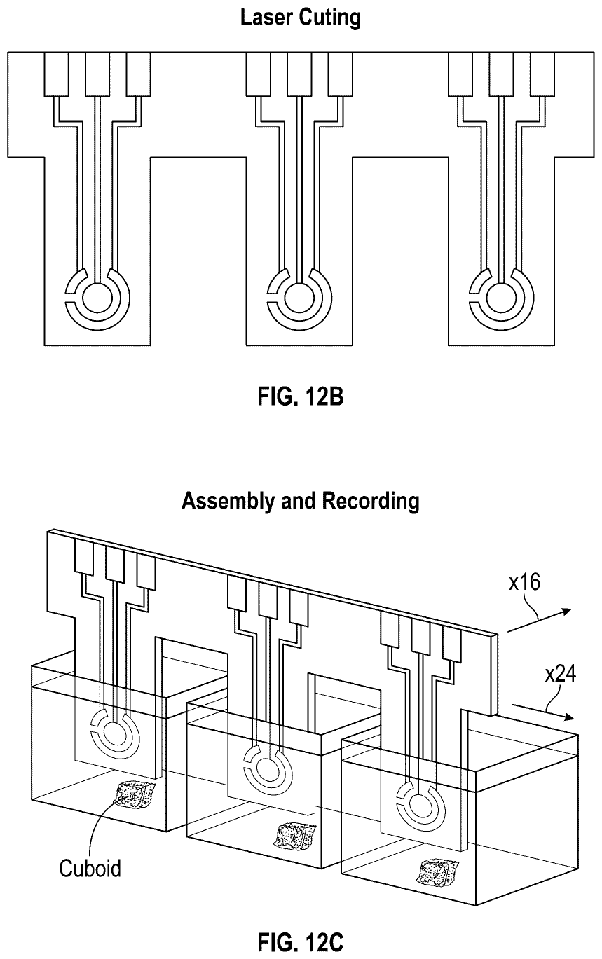

In example embodiments of an integrated E-AB sensor, as shown at FIGS. 12A-12C, a form factor of the integrated E-AB sensor comprises a dip-stick form factor. A dip-stick form factor can enable aptasensors to be arranged in one or more arrays, e.g., in a 16×24 array, as referenced by way of example at FIG. 12C. This arrangement enables a plurality of samples to be monitored in parallel. In addition, a dip-stick form factor can ensure that the solid tissue sample, e.g., as shown at FIG. 12C, does not directly contact the aptasensor, which is immersed in the culture medium.

In embodiments of an integrated E-AB sensor, the nucleic acid aptamer comprises deoxyribonucleic acid (DNA) and the polynucleotide sequence comprises a DNA sequence, and in embodiments, the redox reporter comprises methylene blue (MB). The redox reporter can be linked to the nucleic acid aptamer at a terminus of the nucleic acid aptamer, such as the 3′ terminus of the nucleic acid aptamer.

While the disclosure focuses on Cytochrome C (Cyt-C) as the analyte as an example, other analytes can be used (e.g., with alternate aptamers), without departing from the scope and spirit of the disclosure. In that regard, a polynucleotide sequence can comprise a DNA sequence specific for interaction with Cyt-C, or another analyte or analytes, as the case may be.

In embodiments, the polynucleotide sequence comprises a DNA sequence specific for interaction with Cyt-C. The DNA sequence specific for interaction with Cyt-C can comprise or include a DNA sequence that is at least about 80% identical, at least about 85% identical, at least about 90% identical, at least about 95%, identical, at least about 96% identical, at least about 97% identical, at least about 98% identical, at least about 99% identical, or 100% identical, to the DNA sequence/5′-Thio-C6-Disulfide/CC GTG TCT GGG GCC GAC CGG CGC ATT GGG TAC GTT GTT GC/—NH₂-3′ (SEQ ID NO:1).

In embodiments, the circuitry configured to conduct SWV uses a combined square wave applied to the conductive substrate of the integrated E-AB sensor. In addition, the circuitry can be configured to conduct KDM to determine concentration of the analyte, for example, according to:

$$[ Analyte ] = \frac{K D {M}_{min}}{{( \frac{K D {M}_{max} - K D {M}_{min}}{K D M - K D {M}_{min}} - 1 )}^{n H}}$$

wherein:
[Analyte] is the concentration of the analyte, optionally in ng/mL;
KDMₘᵢₙ is KDM observed in absence of the analyte;
KDMₘₐₓ is KDM expected at saturation of the analyte; and
nH is Hill coefficient.

In embodiments, the circuitry configured to conduct SWV, and/or the circuitry configured to conduct KDM, are implemented as a non-transitory computer-readable storage medium having instructions stored thereon which, when executed by one or more processors, configure the one or more processors to: apply the electric current according to SWV, detect the change in the electric current, and process the change in the electric current according to KDM.

In an aspect, the disclosure provides a method for measuring an analyte with an integrated E-AB sensor. The method comprises: contacting a conductive substrate of the integrated E-AB sensor with a sample comprising an analyte; applying, with circuitry configured to conduct SWV, electric current to the conductive substrate; and processing, with circuitry configured to conduct KDM, a change in an electric current associated with an interaction between an aptamer of the integrated E-AB sensor and the analyte. Methods of the disclosure can be performed, in whole or in part, in any order or sequence of steps, using an integrated E-AB sensor of the disclosure, in embodiments.

In embodiments, the sample comprises a tissue sample within a solution and the analyte is from, or is produced by, the tissue sample. In embodiments, the tissue sample is selected from the group consisting of: a cuboid tissue sample, a tissue slice, an organoid tissue, and any combination thereof. In embodiments, the analyte corresponds with a response of a cuboid tissue sample of the sample to a treatment.

In another aspect, the disclosure provides a kit for measurement of an analyte. The kit comprises: an integrated E-AB sensor, comprising: an aptamer operably connected to a conductive substrate of the E-AB sensor; circuitry configured to conduct SWV to apply an electric current to the conductive substrate; and circuitry configured to conduct KDM to determine concentration of the analyte based on a change in an electric current caused by a conformational change in the aptamer upon an interaction between the aptamer and the analyte. The kit can also comprise instructions for a use of the kit in a method for measuring the analyte with the kit, for example, in the form of printed instructions, a weblink to an internet address containing instructions as text, sound, and/or video, or the like, in embodiments.

In embodiments, the aptamer is operably connected to the conductive substrate and the aptasensor comprises circuitry configured to conduct SWV to apply the electric current to the conductive substrate, and circuitry configured to conduct KDM to determine concentration of the analyte based on changes in the electric current caused by conformational changes in the aptamer upon an interaction between the aptamer and the analyte.

### Kit, Device, System, Circuitry, Processor, and Computer Implementations

Accordingly, in various aspects, all or part of methods, compositions, circuitry, non-transitory computer-readable storage media, instructional materials, and the like, can be integrated into certain commercially-relevant form factors, such as kits, products, devices, computational devices, systems, computational systems, processor-executable code, firmware, software, circuitry, and others.

Accordingly, embodiments of devices and any systems disclosed herein, including embodiments that include or utilize a processor and/or processor executable instructions can utilize circuitry to implement those technologies and methodologies. Such circuitry can operatively connect two or more components, generate information, determine operation conditions, control an appliance, device, or method, and/or the like. Circuitry of any type can be used. In embodiments, circuitry includes dedicated hardware having electronic circuitry configured to perform operations or computations on a dedicated basis, without any use of microprocessors, central processing units, or software or firmware or processor-executable instructions. However, in embodiments, circuitry includes, among other things, one or more computing devices such as one or more processors (e.g., microprocessor(s)), one or more central processing units (CPU), one or more digital signal processors (DSP), one or more application-specific integrated circuits (ASIC), one or more field-programmable gate arrays (FPGA), or the like, or any variations or combinations thereof, and can include discrete digital and/or analog circuit elements or electronics, or combinations thereof.

In embodiments, circuitry includes one or more ASICs having a plurality of predefined logic components. In embodiments, circuitry includes one or more FPGA having a plurality of programmable logic components. In embodiments, circuitry includes hardware circuit implementations (e.g., implementations in analog circuitry, implementations in digital circuitry, and the like, and combinations thereof). In embodiments, circuitry includes combinations of circuits and computer program products having software or firmware processor-executable instructions stored on one or more computer readable memories, e.g., non-transitory computer-readable storage mediums, that work together to cause a device or system to perform one or more methodologies or technologies described herein.

In embodiments, circuitry includes circuits, such as, for example, microprocessors or portions of microprocessors, that require software, firmware, and the like for operation. In embodiments, circuitry includes an implementation comprising one or more processors or portions thereof and accompanying software, firmware, hardware, and the like. In embodiments, circuitry includes a baseband integrated circuit or applications processor integrated circuit or a similar integrated circuit in a server, a cellular network device, other network device, or other computing device. In embodiments, circuitry includes one or more remotely located components. In embodiments, remotely located components (e.g., server, server cluster, server farm, virtual private network, etc.) are operatively connected via wired and/or wireless communication to non-remotely located components (e.g., desktop computer, workstation, mobile device, controller, etc.). In embodiments, remotely located components are operatively connected via one or more receivers, transmitters, transceivers, or the like.

Embodiments include one or more data stores that, for example, store instructions and/or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In embodiments, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In embodiments, circuitry includes one or more user input/output components that are operatively connected to at least one computing device to control (electrical, electromechanical, software-implemented, firmware-implemented, or other control, or combinations thereof) one or more aspects of the embodiment.

In embodiments, circuitry includes a computer-readable media drive or memory slot configured to accept signal-bearing medium (e.g., computer-readable memory media, computer-readable recording media, or the like). In embodiments, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

### Terminology

The description set forth herein in connection with the appended drawings, where like numerals may reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein can be interchangeable with other steps, or combinations of steps, in any suitable combination and/or order to achieve the same or substantially similar result. Generally, the embodiments disclosed herein are non-limiting, and other embodiments within the scope of this disclosure can include structures and functionalities from more than one specific embodiment shown in the figures and described in the specification.

In the foregoing description, specific details are set forth to provide a thorough understanding of example embodiments of the present disclosure. It will be apparent to one skilled in the art, however, that the embodiments disclosed herein can be practiced without embodying all the specific details. In some instances, process steps have not been described in detail in order not to unnecessarily obscure various aspects of the present disclosure. Further, it will be appreciated that embodiments of the present disclosure can employ any combination of features described herein and/or alternatives thereof.

The present application can include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application can also reference quantities and numbers. Unless specifically stated, such quantities and numbers are not to be considered restrictive, but examples of the possible quantities or numbers associated with the present application. Also in this regard, the present application can use the term “plurality” to reference a quantity or number. In this regard, the term “plurality” is meant to be any number that is more than one, for example, two, three, four, five, etc.

As used herein, the term “about,” “approximately,” “near,” etc., includes the stated value as well as non-stated values that are near to or approximate the stated value according to practicable ranges as would be recognized by those skilled in the art. The term “based on” means “based at least partially on.”

In at least some embodiments, “about” refers to the stated value and a range that includes values 10% below the stated value to 10% above the stated value. In embodiments, “about” refers to the stated value and a range that includes values 11% below the stated value, 12% below the stated value, 13% below the stated value, 14% below the stated value, 15% below the stated value, 16% below the stated value, 17% below the stated value, 18% below the stated value, 19% below the stated value, 20% below the stated value, 21% below the stated value, 22% below the stated value, 23% below the stated value, 24% below the stated value, or 25% below the stated value. In embodiments, “about” refers to the stated value and a range that includes values 11% above the stated value, 12% above the stated value, 13% above the stated value, 14% above the stated value, 15% above the stated value, 16% above the stated value, 17% above the stated value, 18% above the stated value, 19% above the stated value, 20% above the stated value, 21% above the stated value, 22% above the stated value, 23% above the stated value, 24% above the stated value, or 25% above the stated value.

In embodiments wherein a range is stated, e.g., the range of 1-16, the stated range includes every value between the lower and upper limits as well as the lower and upper limits of the stated range, themselves, as stated values. In embodiments wherein a range is approximately stated, e.g., about 1-16, the approximately stated range includes every value between the lower and upper limits as well as the lower and upper limits of the stated range, themselves, as stated values (e.g., 1 and 16 are each stated values), including those non-stated values that are near to or approximate the stated values according to practicable ranges as would be recognized by those skilled in the art or as otherwise described herein.

For the purposes of the present disclosure, the phrase “at least one of A, B, and C,” means (A), (B), (C), (A and B), (A and C), (B and C), or (A, B, and C), including all further possible permutations when greater than three elements are listed. Likewise, as used herein, the term “A, B, and/or C” means (A), (B), (C), (A and B), (A and C), (B and C), or (A, B, and C), including all further possible permutations when greater than three elements are listed. Unless otherwise stated, the term “or” is an inclusive “or”, and the phrase “A or B” means (A), (B), or (A and B). Unless otherwise stated, the term “and” requires both elements; for example, the phrase “A and B” means (A and B).

In the claims and for purposes of the present disclosure, the terms “a”, “an”, “the”, and the like, refer to the singular and the plural forms of the object or element referenced. As used herein in the description and claims, the term “comprising”, is inclusive or open-ended and does not exclude additional, unrecited elements or method steps. The term “consisting of,” as used in a claim, excludes any element, step, or ingredient not specified in the claim. The term “consisting essentially of,” as used in a claim, limits the scope of the claim to the specified materials or steps and those that do not materially affect the basic and novel characteristic(s) of the claim.

As used herein, the term “operably connected,” for example, as part of the phrase “an aptamer operably connected to a conductive substrate,” includes any physical connection or coupling, such as a chemical coupling, as described herein, that links the aptamer to the conductive substrate for use. As used herein, “operably connected” includes any such attachments, immobilizations, and the like, for physically coupling the aptamer to the conductive substrate, in various aspects.

### EXAMPLES

### Example 1. Label-Free, Real-Time Monitoring of Cytochrome C Responses to Drugs in Micro-Dissected Tumor Biopsies with a Multi-Well Aptasensor Platform

Functional assays on intact tumor biopsies can complement and extend genomics-based approaches for precision oncology, drug testing, and organs-on-chips cancer disease models by capturing key determinants of therapeutic response, such as tissue architecture, tumor heterogeneity, and the tumor microenvironment. Currently, most of these assays rely on fluorescent labeling, a semi-quantitative method best suited to be a single-time-point terminal assay or labor-intensive terminal immunostaining analysis. In this example, integrated aptamer electrochemical sensors are described for on-chip, real-time monitoring of increases of cytochrome C, a cell death indicator, from intact microdissected tissues with high affinity and specificity, as a non-limiting example of targets able to be monitored with this platform. The platform features a multi-well sensor layout and a multiplexed electronic setup. The aptasensors measure increases in cytochrome C in the supernatant of mouse or human microdissected tumors after exposure to various drug treatments. Since the aptamer probe can be easily exchanged to recognize different targets, the platform can be adapted for multiplexed monitoring of various biomarkers, providing critical information on the tumor and its microenvironment. This approach can not only help develop more advanced cancer disease models but can also be applied to other complex in vitro disease models, such as organs-on-chips and organoids.

### Introduction

As opposed to a modern car or airplane whose state is monitored continuously with hundreds of sensors, most cancer disease models and drug testing platforms do not yield real-time information. In general, information about the state of the tumor is obtained by fluorescent reporters in end-point assays. This shortcoming is especially limiting when the cancer model—typically an animal or a tissue construct—is being exposed to a tumor-killing drug because the drug's pharmacodynamics can have profound effects on its efficacy and toxicity, as well as its ability to amplify its action with other drugs. While many techniques exist to image tumors in vivo, they require complex instruments such as MRI or two-photon microscopes that are not widely available in most laboratories. Microfluidic electrochemical aptasensors for in vivo continuous drug monitoring (MEDIC) can operate without external reagents or imaging equipment, work at room temperature, and can identify various target molecules by exchanging probes. However, their use as in vitro cancer drug probes has only been explored with cell lines and without reporting responses over long periods. Obtaining real-time information about the drug's efficacy on 3D tumor tissue could help provide more precise information on drug effects and insight into the mechanism of action.

In vivo cancer drug testing models result in high attrition rates during clinical development, a failure attributed to inappropriate correlation between the pharmacokinetic and pharmacodynamic parameters, and subsequent extrapolation to human subjects. In the last decade, in vitro models such as patient-derived organoids and organs-on-chips have brought some hope for the development of more precise disease models and more efficient drug testing systems, offering simplicity, scalability, and reproducibility. However, in these platforms, which often take weeks to months to establish, the tumor tissue is generated de novo from the patient's cancer cells or utilizes a synthetic extracellular matrix, generating in vitro cultivation bias. These simplified models lack the 3D structural complexity and the molecular and cellular diversity of the human tumor microenvironment (TME), namely heterogeneous distributions of extracellular matrix (ECM) scaffolds, immune cells, vasculature, and metabolic gradients that can each impact the tumor's response or resistance to cancer therapies. Due to these inefficiencies, only a small percentage (˜14%) of the roughly 1,000 drugs entering clinical trials each year manage to meet the criteria for safety and efficacy, a number that drops to less than 4% for cancer drugs.

Researchers have devised functional platforms for assessing drug responses in live tumor samples. Tumor spheroids, also known as “organoids,” formed from patient-derived, dissociated cells, show promise in replicating in vivo drug responses due to their 3D architecture that recapitulates some cellular and molecular relationships in the TME. However, the steps to create and expand tumor spheroids result in the loss of many native immune cells and their original 3D relationships within the TME, limiting their relevance, especially as models for immunotherapy, since many immunotherapies act on the local TME. Microdissected tumors, derived from cutting tumors into submillimeter tissue pieces, maintain the original TME relatively intact. Implantable or needle microdelivery devices locally deliver small doses of drugs to the tumor in vivo, with maximal preservation of the TME. Still, issues of tumor accessibility and patient safety limit the applicability of implantable approaches. Patient-derived xenograft (PDX) mouse models permit the study of drug responses in an intact organism (including immune checkpoint blockade in humanized PDX), but with the caveat that all or most of the TME is from the host mouse and PDX from individual patients grow too slowly to inform initial post-operative therapeutic decisions. Thus, microdissected tumor biopsies with intact TME can potentially complement and extend genomics- and organoid-based approaches for cancer disease models and drug testing by capturing key determinants of therapeutic response, such as tissue architecture, tumor heterogeneity and the TME. A microfluidic platform based on regularly sized, cuboidal-shaped micro-dissected tumor fragments (referred to as “cuboids”) that are mechanically cut with a tissue chopper can enable more than 10,000 cuboids (˜400 μm-wide) to be produced from ˜1 cm³ of solid tumor; the cuboids are never dissociated and retain much of the native TME, including viable immune cells and vascular structures.

However, despite the great potential of functional testing platforms for broad disease modeling and drug screening applications, accurately characterizing the behaviors of microdissected tumors and their interactions with drug compounds can be challenging. The levels of secreted biomarkers from tissues serve as key indicators of their dynamic status, changing as they grow, mature, and undergo external stimuli or damage. A significant challenge of current functional assay platforms lies in their limited ability to collect real-time data about the tumor's secretome as an assessment for drug screening and probing disease pathologies. At present, most of these assays rely on off-chip or fluorescent labeling analysis, including enzyme linked immunosorbent assay (ELISA) and Luminex assay (which are quantitative methods best suited to single time-points) or immunostaining analysis (a terminal, labor-intensive, and poorly quantitative assay).

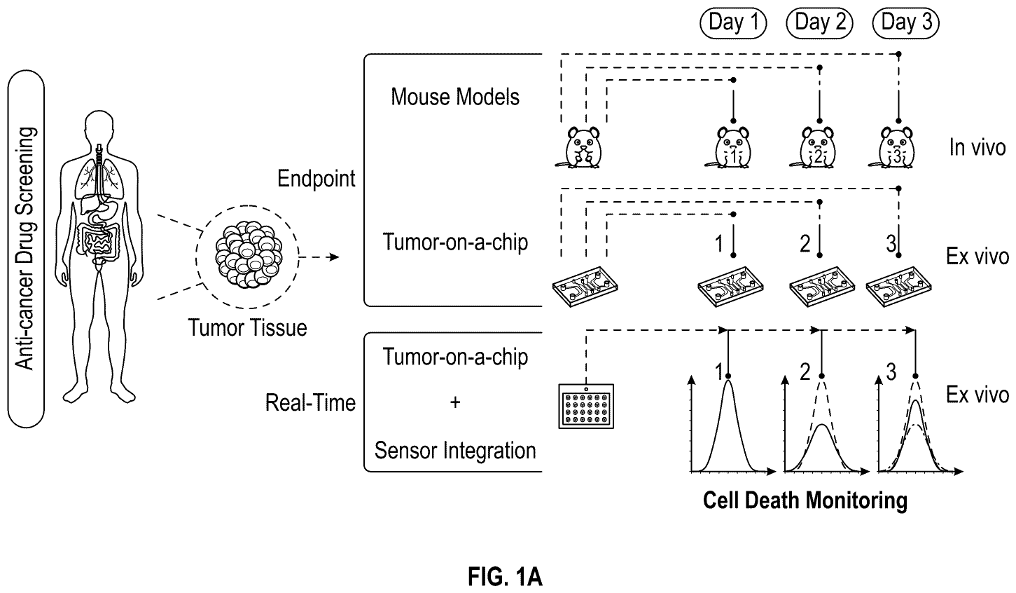

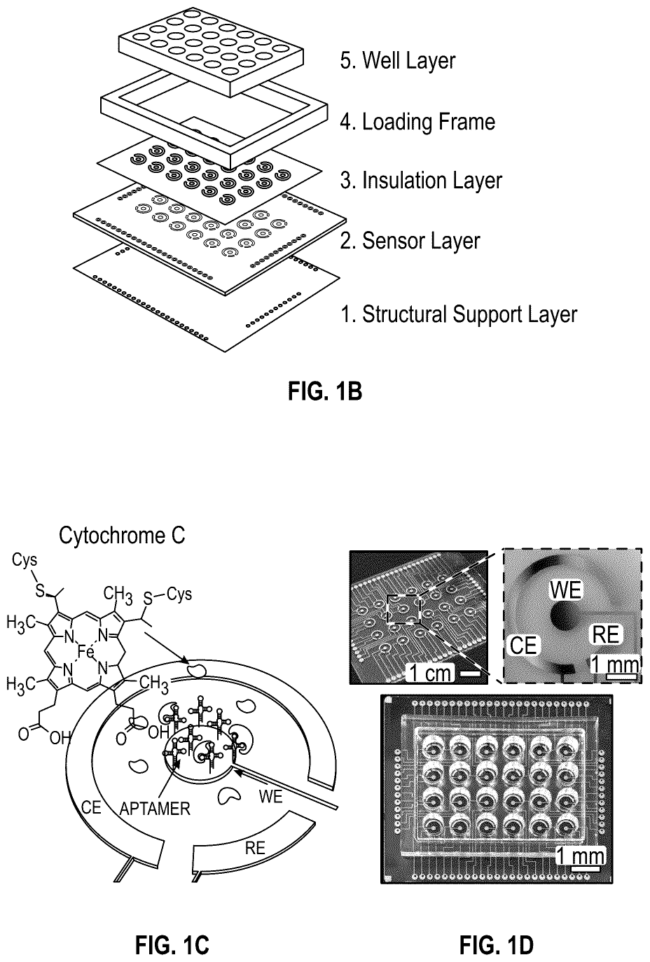

Aptasensors employ nucleic acid aptamers to directly measure ligand binding. The aptamers undergo reversible conformational changes when binding to their molecular targets. The electrochemical signal response reflects changes in distance between an electrochemical reporter covalently linked to the aptamer and the gold sensor surface upon binding. This biosensor transforms aptamer-target interactions into an electrically measurable signal, enabling cost-effective, on-chip monitoring of specific biomarkers. Aptasensors offer distinct advantages, as they eliminate the need for processes like washing, separations, chromatographic steps, or costly equipment. Moreover, this technology has been adapted to monitor drug levels or relevant biomarkers, such as cytokines and other physicochemical parameters from tumor functional testing platforms. Additionally, Kinetic Differential Measurement (KDM) techniques have been developed to address baseline drift (crucial for extended measurements in a cultured environment and accurate calibration), enhance the signal-to-noise ratio, and mitigate variations between aptasensors. The integration of biosensors into functional platforms can offer noninvasive, real-time monitoring of relevant biomarkers in situ. An electrochemical aptamer-based sensor (“aptasensor”) is incorporated into a cuboid platform to monitor the secretion of cytochrome C (CytC), a cell death indicator, in real time. This mitochondrial redox enzyme is released into the supernatant upon apoptotic cell death. Because the aptamer can be switched to detect any other secreted compound, the approach can be generalized to any other secreted molecule for which there exists an aptamer. One can implement a CytC aptamer for electrochemical sensors to detect cell death, possibly as a single-time-point measure and in blood serum samples. The disclosed example platform enables on-chip real-time monitoring of the dynamic response of cuboids and their intact TME to pharmaceutical compounds, as drugs can trigger acute, chronic, and/or delayed cellular responses from both tumor cells and nontumor (e.g., immune) cells. This device enables to understand the temporal dynamics of cellular responses to treatments in intact tissue and thereby shed light on predicting and determining the response of cancer to treatments (FIG. 1A). Complementing the platform's capability, a multiplexer system for a single-channel potentiostat is disclosed, featuring a printed circuit board (PCB) that interfaces with the electrochemical sensor platform. The multiplexer streamlined the process by reducing costs and simplifying the procedure of reading from multiple electrodes, ultimately enhancing throughput, user-friendliness, and cost-effectiveness, which is particularly valuable given the high expense of amplifiers. With the disclosed sensor platform, CytC is detected in the supernatant of mouse or human cuboids that had been exposed to drug treatments. This verification demonstrates the disclosed platform's ability to detect various relevant biomarkers secreted from intact biopsies. This platform can help to better evaluate targeted therapies and immunotherapy combinations with other drugs, an emerging challenge given the vast number of potential drug combinations and the limited resources for testing.

### Results

Design and fabrication of the multi-well platform with an integrated microelectrode sensor array. A custom multielectrode cell culture plate with integrated aptasensors was designed and fabricated. The custom plate included 24 wells, mirroring the format of a quarter of a standard 96-well plate. It featured 6 columns and 4 rows, with a well-to-well spacing of 9 mm.

Methods were adapted from a microfluidic multi-well platform for drug testing of cuboids. The platform (FIG. 1B) is made in poly (methylmethacrylate) (PMMA) layers by digital manufacturing using CO₂ laser micromachining, solvent bonding, and transfer adhesive techniques. The system contains five PMMA layers: (1) a structural support layer; (2) a sensor layer containing the sensing electrodes (fabricated by a combination of photolithography, dry film photoresist technology, and hot roll lamination) on top of an optional microfluidic channel layer; (3) an insulation layer made of removable 3M PolySil silicon adhesive that surrounds the sensor's working areas and prevents electrical shorting; (4) a frame that forms an outer border; and (5) a bottomless 24-well plate with removable 3M PolySil silicone adhesive (3M300LSE) for the culture stage. The sensor layer includes an array with 24 sets of bare gold (Au) microelectrodes. Each well has one set of three electrodes: a working electrode (WE), a reference electrode (RE), and a counter electrode (CE). The sensor fabrication process is further detailed herein. For the base of the aptamer working electrode, bare Au was used or another layer of AuNPs formed. For the counter electrode, a bare Au electrode was used directly. For the reference electrode, another layer of Ag/AgCl was added. In the last step, CytC aptamers were added onto the working surface using thiol bonds (FIGS. 1C-1D).

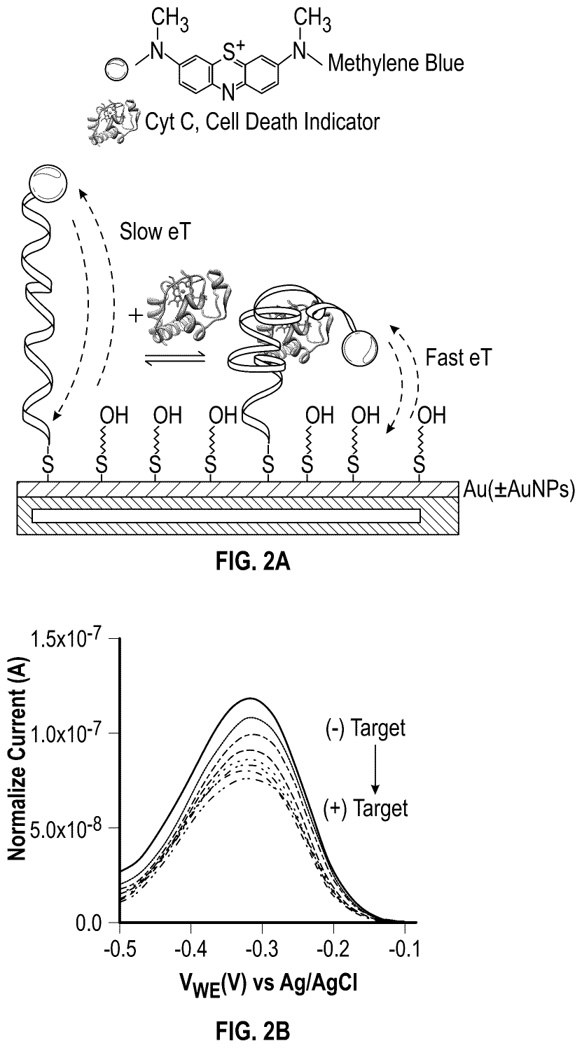

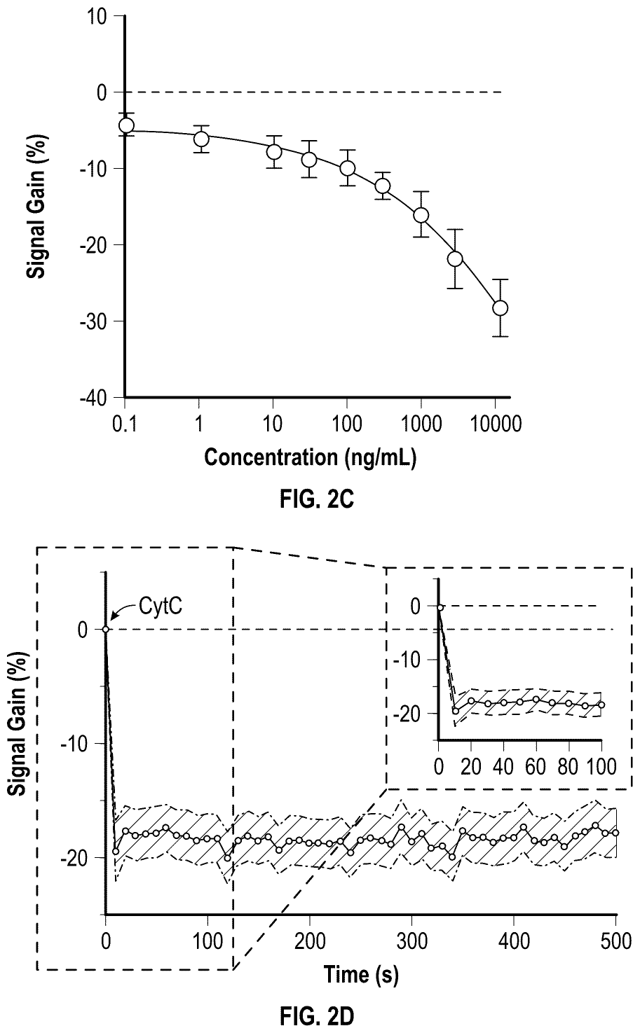

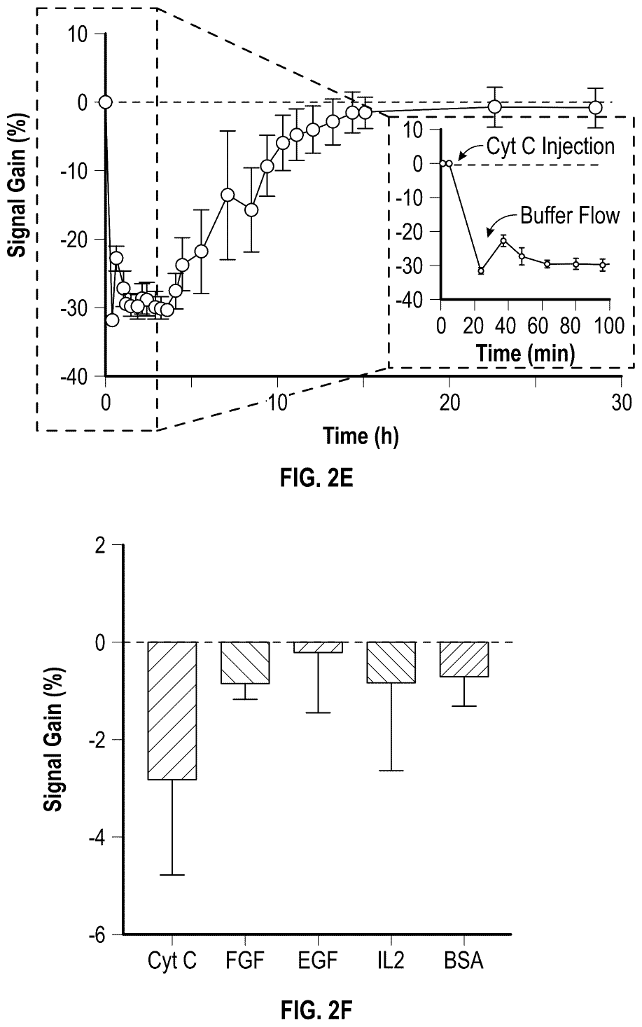

Characterization of the electrochemical aptamer-based sensor. An electrochemical sensor with a modified aptamer that recognizes CytC was developed, enabling real-time monitoring of CytC increases in the supernatant due to cell death in cuboids during drug screening. The aptamer sequences were modified with a redox-active molecule, methylene blue (MB), at the distal end of the aptamer. As shown at FIG. 2A, the binding of the CytC target induces a rapid and reversible conformational change in the aptamer, increasing the electron transfer (eT) rate between the MB reporter and the WE surface. This change in eT results in a measurable variation in redox current, easily detectable using square wave voltammetry (SWV). Changes in peak current height were monitored, which correlates with the proximity of the MB redox tag to the electrode. The performance of the aptasensors in response to varying concentrations of CytC (ranging from 1 to 30,000 ng/mL) is presented at FIG. 2B. At a recording frequency of 50 Hz, the peak current height of the sensors decreases as the CytC concentration increases. As shown at FIG. 2C, a calibration curve measured at 50 Hz allowed to estimate an apparent dissociation constant, KD, of 6.19±1.57 ng/mL (515±130 μM) through nonlinear regression fitting to the Langmuir-Hill isotherm. This KD of ˜6 ng/mL, with a dynamic range up to 39,580 ng/mL, indicates the strong binding affinity of the aptasensors for CytC and its ability to detect relevant concentrations (up to 3.32 μM).

Binding kinetics of the CytC aptasensor. The binding kinetics of aptamer-protein complexes were further evaluated. Studies looking at the signal in response to a constant amount of CytC ligand indicated that the aptamer-target binding required at least 10 see to establish equilibrium (FIG. 2C). Next, the monomolecular dissociation rate constant (k_{off}) was computed. The aptasensor platform was adapted for flow injection analysis under constant flow. The sensor platform was modified to incorporate an inlet and outlet loop injection system. With this setup, the different phases of aptamer-target interaction were able to be measured, including association, dissociation, and equilibrium. FIG. 2E shows the current response during the injection of the targets. Initially, the aptamer remained unbound in the absence of the target, producing baseline levels of current. The introduction of the target initiated the association phase, which led to the occupation of aptamer binding sites. This phase continued until a steady-state or equilibrium point was attained. Subsequently, the semi-automated target removal initiated the dissociation phase of the aptamer-target complex. The continuous flow of the medium enabled the aptasensor to recover eventually, gradually returning to the baseline levels within approximately 12 hrs.

Selectivity of the CytC aptasensor. Selectivity is a factor for the practical application of these sensors in tumor-on-a-chip models for drug screening. To assess the selectivity of the sensors, experiments were conducted in which the sensor's responses to CytC (100 ng/mL) vs. responses in the presence of other potentially interfering molecules (also at 100 ng/mL) were measured. Measurements were performed in a growth medium at 50 Hz. Under these conditions, CytC leads to a higher signal gain compared to others such as human fibroblast growth factors (FGF), epidermal growth factor (EGF), interleukin-2 (IL-2), and bovine serum albumin (BSA) that might be found in culture medium or secreted from tumor tissues (FIG. 2F).

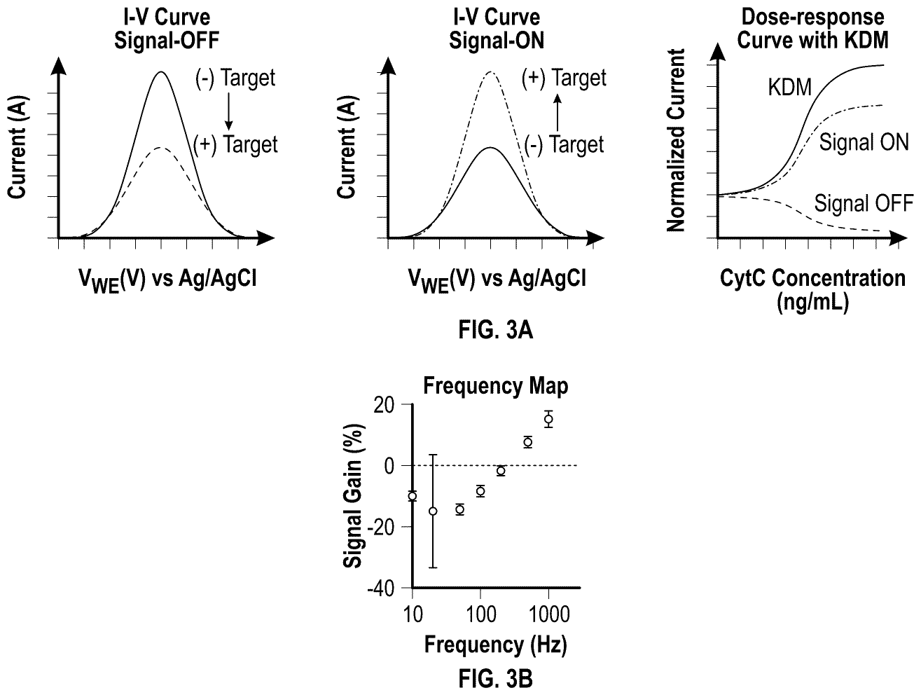

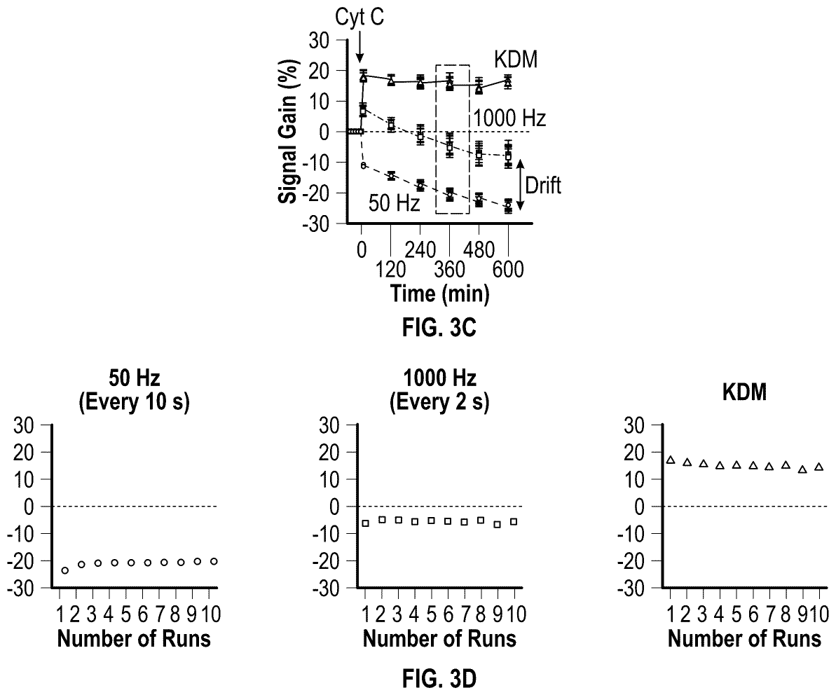

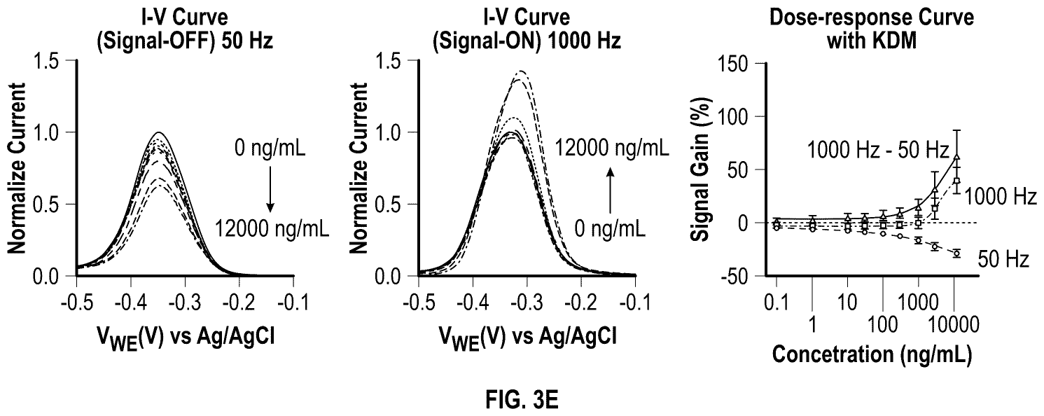

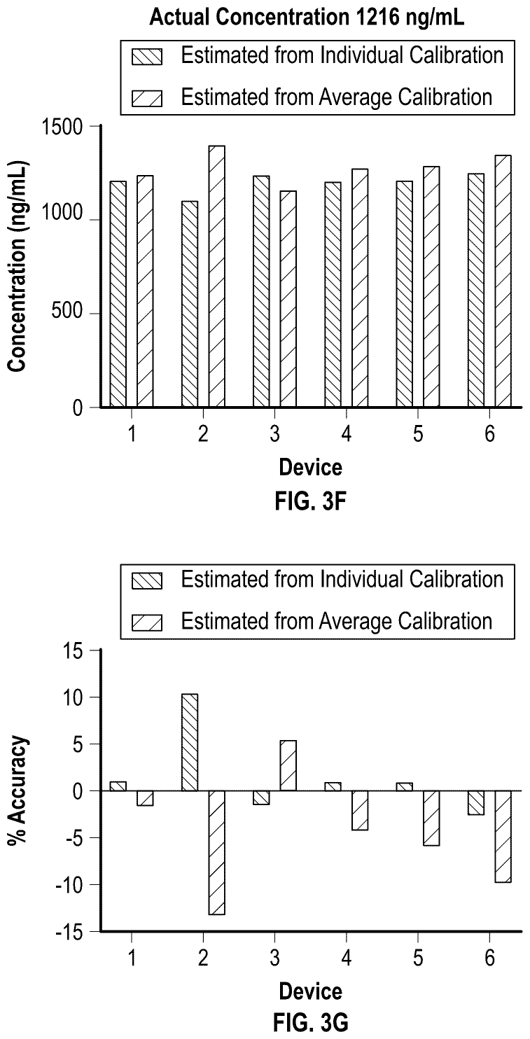

Kinetic Differential Measurement (KDM). To correct for baseline drift (a factor for measurement over time in culture and accurate calibration), enhance signal-to-noise, and reduce sensor-to-sensor variability, an approach that uses the difference between signal-ON (increase current) and OFF (decrease current) curves at different frequencies was innovatively implemented. This approach, called Kinetic Differential Measurement (KDM), can be used for in vivo electrochemical aptasensors that detect real-time drug levels. KDM measures the signals at two different states of the electron transfer at the WE surface. At the fast signal-ON state, the electrochemical signal increases with target addition. At the slow signal-OFF state, the electrochemical signal decreases with target addition. Taking the difference between the two measurements, one can improve the gain of CytC sensors and correct the baseline drift (FIG. 3A).

Conditions to perform KDM with the CytC aptasensors were determined. Since the relative change of this current is highly dependent on SWV frequency, the sensor was interrogated at multiple SWV frequencies. Their amplitude was plotted between no target and saturation to generate a frequency map (FIG. 3B). A signal-OFF response was observed from most of the frequencies tested, from 10 Hz to 200 Hz, and a signal-ON response at 500 Hz and 1,000 Hz. The amplitude-frequency pairs that achieved the most significant signal difference between no target and saturation were observed. As a potentiostat can potentially require more than 50 see to perform a SWV scan at 10 Hz and generate noisy currents at 20 Hz, 50 Hz was selected for the signal-OFF frequency and 1,000 Hz for the signal-ON frequency for CytC measurements. Using the previous parameters, the stability of KDM recordings was evaluated. With KDM, one could differentially combine the two output signals to reduce background noise and drift. The sensors were exposed to a CytC concentration of 2000 ng/mL in culture medium (DMEM-F12-10% fetal bovine serum (FBS)) (FIG. 3C) or to Phosphate-Buffered Saline (PBS) for 10 hrs. Data was collected every 2 hrs with 10 SWV scans each time. The use of KDM reduced the drift when measuring at 50 Hz and 1,000 Hz alone in both cases. There can be two potential distinct mechanisms contributing to signal drift. The first exponential phase involves fouling by culture medium components. The observed drift was more pronounced due to fouling from interferents, such as proteins absorbing to the sensor surface, when measuring in the culture medium (FIG. 3C) compared to measuring in PBS. With KDM, the signal remained stable over 10 hrs in the culture medium. The second phase of signal drift arises from the loss of reporter-modified DNA due to electrochemistry desorption of the monolayer. Thus, there is a tradeoff between the frequency measurements and the duration of the experiment. Therefore, data collection was limited to ten repeated runs every two hours at 50 Hz and 1,000 Hz, for a total of ˜120 runs per sensor. This approach resulted in a stable signal, and any residual drift was effectively corrected using KDM (FIG. 3C-3D).

Optimization of the gold electrochemical aptamer biosensor surface. The response of a conventional planar Au surface was evaluated. The Au-aptasensor setup exhibited a robust signal response, characterized by well-defined MB reduction peaks, across the relevant human CytC recombinant concentration range of 0-12,000 ng/mL (FIG. 3E). The Au-aptasensor configuration offered the benefit of straightforward replication and minimized variability, making it the preferable choice for these example monitoring objectives. In the exploration of alternative approaches, integrating gold nanoparticles (AuNPs) onto planar Au surfaces before the aptamer immobilization process to enhance the signal-to-noise ratio (SNR) and overall working electrode quality was considered. The AuNPs-aptasensors indeed exhibited higher current responses and displayed a larger-amplitude dose-response curve with average KDM values compared to Auaptasensors. However, the fabrication of AuNPs-aptasensors led to variability between sensors, rendering it unsuitable for at least some multielectrode settings. Nevertheless, these AuNPs-aptasensors can enable studies requiring fewer sensors and higher sensitivity for measuring lower-abundance biomarkers at low pg/mL levels. Despite the higher sensitivity of the AuNPs-aptasensor, the Au-aptasensor configuration was chosen for all experiments due to its suitability for these example monitoring requirements, ease of replication, and the assurance of consistent and reliable results, particularly in rapidly detecting CytC at a higher concentration range. To ensure a high-quality electrode surface, an alternative cleaning protocol was employed, compared to the conventional cleaning method, with hydrogen peroxide (H₂O₂) and linear sweep voltammetry (LSV) in potassium hydroxide (KOH), which is compatible with the PMMA based platform. This method effectively prepared a clean Au surface for subsequent processing, allowing for strong covalent binding of aptamers and efficient signal collection for the aptasensors. The cleaned sensors exhibited distinct MB reduction peaks, indicating the formation of a compact self-assembled monolayer. In addition to Au surface cleaning, the challenge of generating a stable Ag/AgCl film for the on-board reference electrodes was addressed. Traditional methods were not feasible due to the PMMA substrate. Instead, an alternative protocol involving Ag plating was adopted, as well as chemical cleansing, cyclic chlorination, improved interfacial adhesion, and a final Nafion layer. This approach provided a thin yet robust Ag/AgCl/Nafion electrode surface, ensuring stability and longevity for the reference electrodes. A detailed description of the entire process can be found herein.

Aptasensor variability. The variability in sensor performance using KDM was assessed. Six individual sensors were calibrated at 50 Hz and 1,000 Hz with a concentration range of 0-10,000 ng/mL using human CytC recombinant in culture medium (DMEM-F12-10% FBS). Then, these calibrated sensors were used to measure a known concentration of CytC and the actual concentrations compared to the obtained values that were determined from the dose-response curve based on the KDM values. Accuracy, defined as the mean of the relative difference between the estimated and applied concentrations (100×(expected—observed)/observed) based on a published protocol, was 1.48% when calibrating each sensor to itself, with a coefficient of variation (100×population standard deviation/population mean) of 5.28%. However, using an average calibration derived from all six sensors, the mean accuracy slightly increased to 4.29%, but remained within an acceptable range. The coefficient of variation was 6.78%, indicating a marginally increased variability when applying the average calibration (FIGS. 3F-3G). These results demonstrate low variability within the sensors from the same batch, confirming their suitability for reliable monitoring.

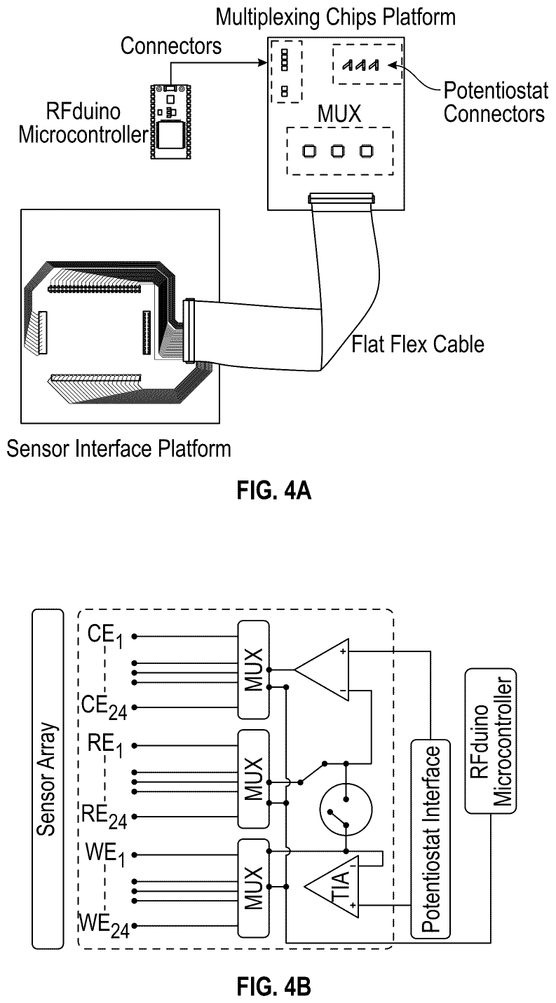

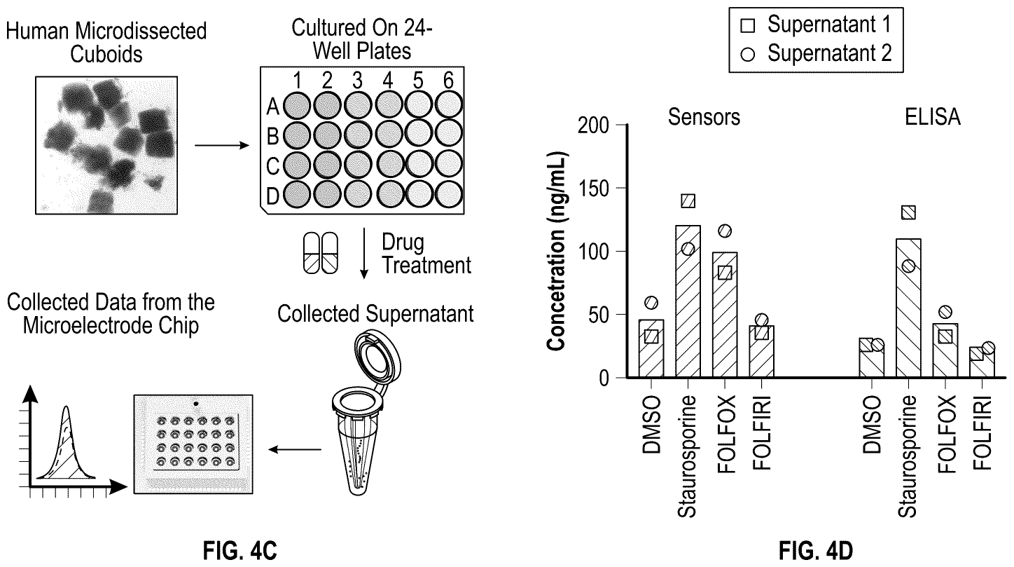

Automated switching between electrodes using an electronic multiplexer. Electrochemical potentiostats are expensive. To reduce the cost and simplify the procedure of reading from multiple electrodes, an electronic multiplexer setup that allowed for the automated switching of a single potentiostat to each of the 24 sensors was implemented. The reduction in bandwidth does not affect the final measurements because CytC secretion is a slow process. The system included a customized PCB board to plug in the sensors and three 32-to-1 channel multiplexer chips (one for the three sets of WE, RE, and CE electrodes), all situated on a separate PCB board. A flat flex cable (FFC) connected the PCBs and allowed to separate the sensor platform board as a stand-alone unit for incubation in a 37° C. incubator. Furthermore, communication was established between the electrode array and a single-channel commercial potentiostat (DY-2219, Digi-Ivy) by connecting the multiplexing board with a HiLetgo microcontroller (FIG. 4A). This setup enabled electronic switching between various electrodes and enhanced throughput for drug applications on microdissected tissues, offering significant advantages over single-working electrode-based electrochemical detection platforms. FIG. 4B illustrates the hardware block diagram for the multiplexing platform from the sensor arrays through the multiplexers to the potentiostat. The effect of the multiplexer on the electrochemical signal acquired from the sensor platform was evaluated. The SWV results obtained directly through a single-channel potentiostat and the SWV results obtained indirectly through the multiplexer interface were compared. Both conditions exhibited similar responses, with less than 5% difference in normalized peak values at 50 Hz and 1,000 Hz across the tested devices.

Measurement of CytC from the supernatant of human microdissected cancer tissue cultured with drugs using the aptasensor platform. A study was conducted to assess the aptasensor platform's ability to quantify CytC secretion from human cuboids after drug treatments for three days in culture with Staurosporine, FOLFOX (a combination of fluorouracil and oxaliplatin), and FOLFIRI (a combination of fluorouracil and irinotecan), in comparison to the dimethyl sulfoxide (DMSO) vehicle control. Uniform-sized cuboids were prepared from a human colorectal tumor biopsy. Each drug condition was prepared in duplicate. Subsequently, the supernatants were collected and the level of CytC released from the cuboids after cell death was measured using the aptasensor platform. To validate the platform's measurements, the results were compared with those obtained from ELISA (FIG. 4D). The aptasensor platform showed a marked increase in the CytC production with Staurosporine treatment (˜3-fold) compared to the DMSO control vehicle. In contrast, compared to the DMSO control vehicle, a more modest increase (about 2.25-fold) was observed with FOLFOX treatment and a slight decrease (0.92-fold) in CytC production was observed with FOLFIRI treatment. The trend from ELISA results closely follows this observation, with CytC production increasing by 4.23-fold for Staurosporine, 1.6-fold for FOLFOX, and a decrease to 0.73-fold for FOLFIRI, all relative to the DMSO control vehicle, based on duplicated supernatant samples. In some cases, the platform even indicated higher production rates. Duplicates for both the sensor and for the ELISA were consistent. These results aligned overall with those from ELISA, confirming the aptasensor's capability to measure CytC production from human cuboid samples in real time. However, a slight discrepancy in CytC concentration was observed with FOLFOX treatment. While the trend in measurements for supernatant 1 and supernatant 2 was similar between sensor reading and ELISA, the higher concentration observed in sensor readings may stem from variations in the sensor-specific calibration curves.

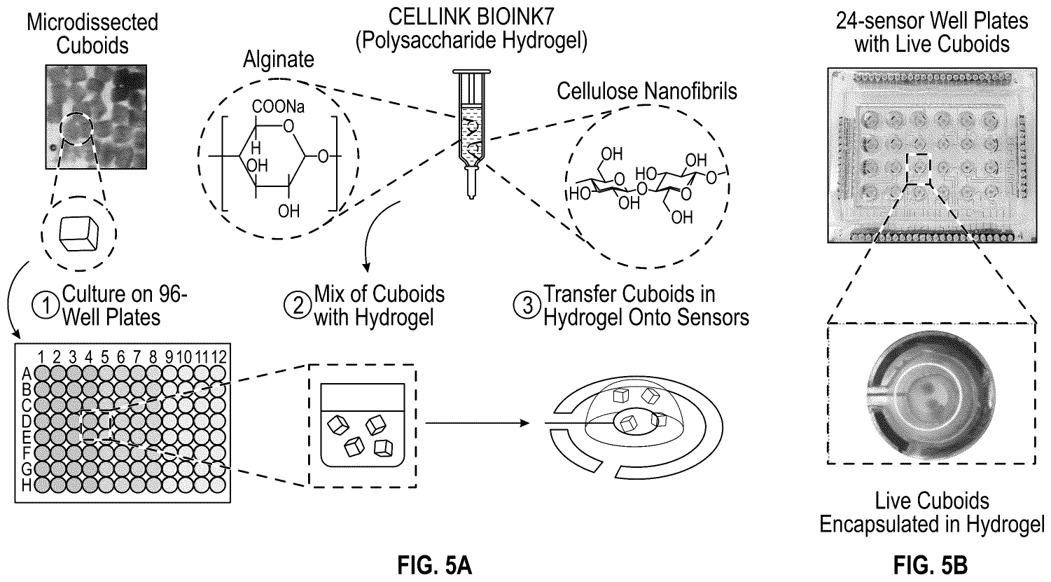

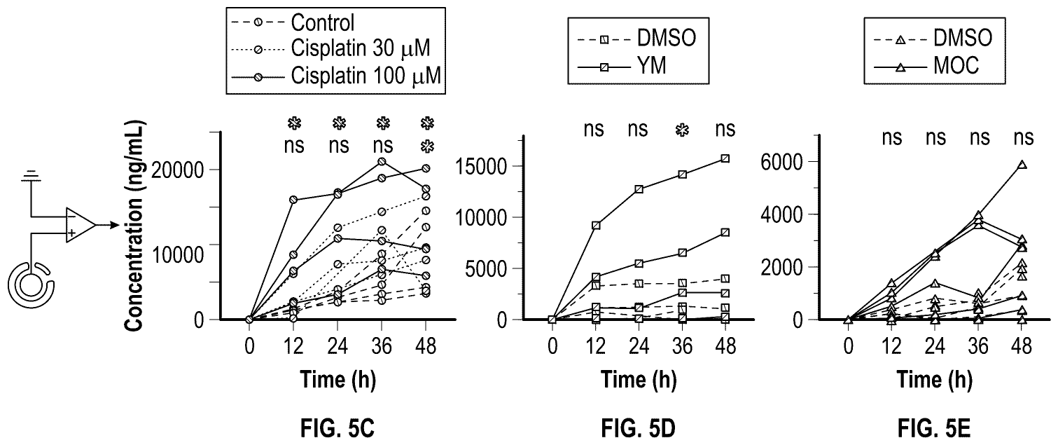

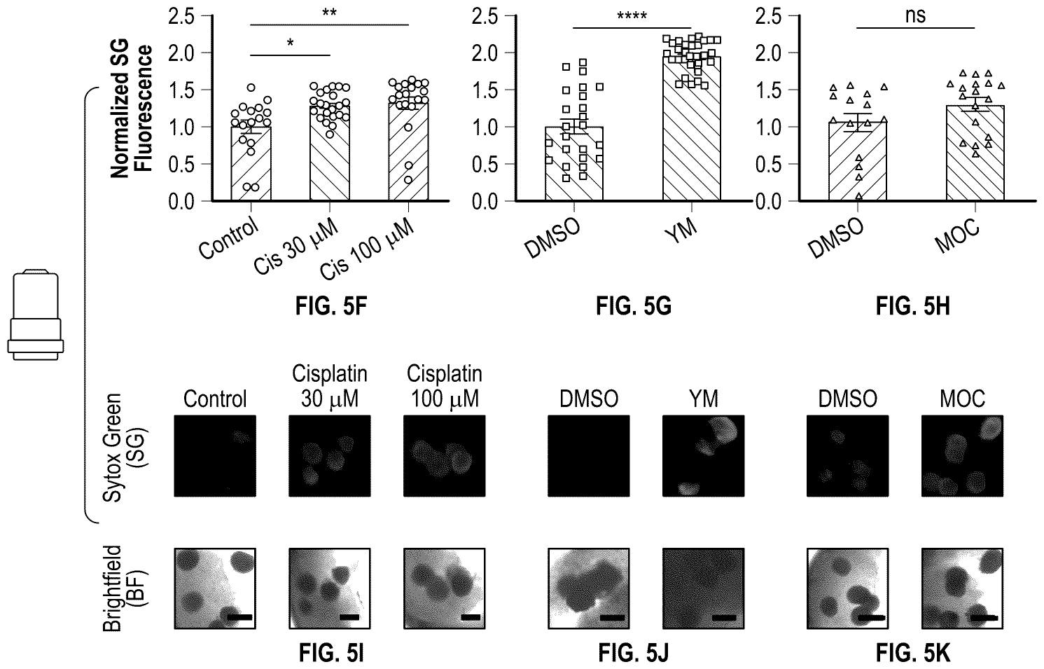

Ex-vivo monitoring of CytC from microdissected tumor tissues after drug treatment in culture using the aptasensor platform. The sensor platform's ability to quantify CytC secretion from cuboids was evaluated at different time points during an extended culture period (48 hrs). Cuboids from human U87 glioma cell-derived tumors grown in athymic nude mice were prepared. After overnight culture in a 96-well plate to allow recovery from tissue damage due to the cutting procedure, the cuboids were transferred onto the sensor platform for measurement. To prevent random adhesion of the cuboids to the sensor surface and improve the precise localization of CytC secretion detection, the cuboids were encapsulated in a hydrogel. They were mixed with hydrogel before transferring onto the sensors and immobilizing them to a restricted area just above the working electrode (FIGS. 5A-5B). The hydrogel matrices exhibited interconnected pores and good water retention, thus maintaining a suitable environment for tissue proliferation and growth. Encapsulation of cells in hydrogels in close proximity to the WE also generates a more localized and distinguishable electrochemical response. Hydrogels can be used as a protective layer to ensure the long-term stability of aptasensors. A polysaccharide hydrogel (Cellink, Bioink, Boston, USA) that contains nanofibrillar cellulose and sodium alginate was used. It was found that placing this polysaccharide hydrogel atop the working electrode (WE) did not hinder the sensor's ability to detect CytC and perform KDM. It was also observed that the performance of the sensors remained unhindered regardless of the hydrogel concentration, apart from an extension of the reaction time to approximately 30 min. However, it was also found that a 100% hydrogel concentration posed challenges during the transfer of encapsulated cuboids to the surface, potentially resulting in the inadvertent removal of electroactive aptamers during the transfer. As a result, the hydrogel concentration was diluted to 50% with Dulbecco's Phosphate-Buffered Saline (DPBS), permitting effective transfer while ensuring proper hydrogel adhesion to the electrode surface. The natural hydrogel collagen was not used, because its application on top of the electrodes impaired the detection of CytC and did not allow for KDM. Next, the sensor platform's ability to measure CytC secretion from cuboids in response to exposure to cytotoxic drugs was evaluated. Initially, the baseline signal was measured with the encapsulated cuboids on the WE before exposing U87 cuboids to different concentrations of cisplatin (a cytotoxic chemotherapy drug, FIG. 5C), sepantronium bromide, (YM-155, a survivin inhibitor and potential anti-cancer drug, FIG. 5D), and mocetinostat (MOC, a histone deacetylases inhibitor cancer drug, FIG. 5E) for 48 hrs. For cisplatin, six wells were treated, each at a different concentration (0, 30, 100 μM), the sensor signal collected every 12 hrs. For YM-155 and MOC, eight wells were treated, each at 1 μM and 10 μM, respectively, compared with DMSO vehicle control, and the sensor signal collected every 12 hrs. Untreated cuboids exhibited lower CytC values from 12 to 48 hrs. Tissues cultured with 100 μM cisplatin exhibited the highest levels of CytC, beginning at 12 hrs and showing incremental growth over the 48-hr period. The cuboids cultured with 30 μM cisplatin displayed a higher CytC level compared to the control but with a different time course. CytC levels increased in more pronounced increments from 12 to 24 hrs and 24 to 36 hrs, followed by marginal growth from 36 to 48 hrs. Notably, the result closely resembled the range observed in tissue treated with 100 μM cisplatin at the 48-hr mark. Additionally, experiments were conducted in which the sensors' responses to CytC (100 ng/mL=8.3 nM and 2000 ng/mL=166 nM) versus responses in the presence of all the drugs used to treat the microdissected tumor tissues, ensuring the selectivity of the sensors in these conditions. Measurements were performed in a culture medium at 50 Hz and 1,000 Hz and KDM signals generated from measurements at 50 Hz and 1,000 Hz. Under these conditions, CytC responses led to a higher signal gain compared to the responses in the presence of relevant drug concentrations, such as DMSO control vehicle (0.2%), 10 μM MOC, FOLFOX (1 g/mL 5FU+1 μg/mL Oxaliplatin), 30 μM and 100 μM Cisplatin, 1 μM Staurosporine, and 1 μM YM-155. Terminal live/dead staining of the cuboids was also performed (FIGS. 5F-5K) to confirm the electrochemical obtained with the sensor platform (FIGS. 5C-5E). After 48 hrs in culture, the cuboids were stained with the green fluorescent dead nuclear stain, SYTOX green (SG), and the blue fluorescent pan-nuclear stain, Hoechst (H) (FIGS. 5F-5K). Control cuboids displayed lower SG dead stain fluorescence (normalized to the mean), whereas treated cuboids with 30 and 100 μM cisplatin exhibited higher SG dead stain fluorescence. The findings indicated a statistically significant response to 30 μM and 100 μM cisplatin (FIGS. 5F, 5I). These observations were consistent with fluorescence-based findings and agreed with the sensor-based responses. This endpoint live/dead analysis confirmed the CytC aptasensor cell death responses to various cisplatin concentration treatments. Similar experiments were also performed with the CytC aptasensors cuboids treated with other chemotherapy drugs, YM-155 (FIG. 5D) or MOC (FIG. 5E) versus DMSO vehicle control. Similar to cisplatin, it was observed that the YM-155 treatment also showed cell death responses by increases in CytC. The treatment with YM 155 exhibited a higher CytC production rate than the DMSO control, first seen at 12 hrs and displaying incremental growth over the 48-hr period. The terminal live/dead staining revealed elevated SG dead stain fluorescence in cuboids treated with YM-155 compared to the DMSO control (FIGS. 5G, 5J). Finally, a similar trend was noted with the treatment involving MOC, though at much lower levels and not statistically significant. The MOC-treated cuboids exhibited a higher average CytC production rate than the DMSO control. However, the sensor data indicated that the variance in CytC secretion levels between MOC-treated cuboids and the DMSO control was less pronounced compared to the difference observed in the YM-155 treatment. These findings were substantiated by the cell live/death staining, which revealed slightly elevated SG dead stain fluorescence in cuboids treated with MOC compared to the DMSO control (FIGS. 5H, 5K).

### Discussion

An integrated electrochemical sensor platform that enables on-chip, real-time monitoring of cell death by increases of CytC in the medium of long-term cultures of intact tumor biopsies is described herein. These measurements were made with a multi-well sensor layout and a multiplexed electronic setup. With this platform, the in-situ response of cuboids to various chemotherapy treatments was measured. The sensors demonstrated selectivity, specificity, and reproducibility while minimizing interference. The aptasensors are also able to effectively utilize Kinetic Differential Measurement (KDM), a technique specific to the aptamer used, to address signal drift caused by long-term culture. The temporal sensitivity of the CytC aptasensor results from the binding kinetics of the CytC aptamer receptor, with a fast ON response (seconds) and a slow OFF response (hours). A fundamental tradeoff between thermodynamic characteristics (sensitivity) and kinetic properties (time resolution) actively influences molecular interactions. In situations such as this example, with high-affinity receptors and low target concentrations, achieving equilibrium between the receptor's bound and unbound states takes longer due to a slow k_{off} rate. These kinetics introduce a slight delay in generating the final sensor readout. This characteristic is evident in this case, where a higher receptor affinity results in a slower off-rate (k_{off}=12 hrs). This slow timescale of OFF measurements is compatible with the goals for a CytC cell death sensor. Cell death markers like CytC are not continuously released, but released during specific events, such as apoptosis induced by chemotherapy drugs. This transient secretion pattern means that CytC concentration in the culture medium can fluctuate over time. Because of the slow decrease in signal over hours, one can be confident to not miss increases in CytC that may have occurred between readings, which can be taken periodically. Thus, stable binding to the sensor surface is a factor, enabling to track dynamic increases in CytC levels while minimizing background noise. One can allow the sensor more time to reach equilibrium to address the slow equilibration kinetics. Consequently, the slow, high-affinity sensor enables to measure low-abundance analytes in the nM range over hours, compared to other, real-time, small-molecule sensors (e.g., for drugs) that measure higher abundance analytes in the M range over seconds/minutes. The integrated sensor platform, as demonstrated by ex vivo characterization results, effectively enables to track CytC release over time under drug treatments. The platform reliably yields high SNR ratio measurements during extended ex vivo operations (up to 48 hours). These experiments demonstrated the capability of the Au planar sensors to achieve high SNR measurements of CytC over prolonged periods in the culture medium. Furthermore, the successful fabrication of AuNPs with an increased surface area for aptamer immobilization positions the platform for future applications, particularly in measuring lower-abundance biomarkers like cytokine biomarkers at the pg/mL level.

In this example, the utilization of hydrogel to prevent fouling and improve the precise localization of CytC secretion detection was established. The choice of hydrogel can affect the performance of the aptasensor. The electrochemical aptasensor relies on biorecognition elements, including the incorporation of a redox probe at the 3′ distal end of the oligonucleotide, and the resulting signal depends on a conformation change of the surface-bound aptamer. As sensor signaling relies on aptamer conformation and flexibility, the aptamers can be able to move freely upon target binding to generate electron transfer between the redox probe and the electrode surface for signaling. Hence, it is a factor to ascertain that the hydrogel does not appreciably impact the signaling capacity of the sensors and that the sensors can quantitatively respond to CytC at varying frequencies to ensure effective utilization of KDM. Collagen matrices were first considered, since they are commonly utilized for cell culture and microencapsulation of proteins or small molecules for drug delivery. Unfortunately, collagen encapsulation obstructed the aptasensor's ability to perform KDM, even at half strength. Moreover, collagen led to a prolonged reaction time, preventing the sensor from reaching an equilibrium state even after 90 minutes. Without wishing to be bound by any particular theory, it can be hypothesized that the density of the collagen gel partially impairs the conformational change of the aptasensor upon binding. These limitations prompted to explore alternative hydrogels. One alternative, Cellink bioink, exhibited successful sensor performance. This finding underscores the factor of material selection for sensor signaling capacity.

Despite successful periodic monitoring and discrimination of CytC secretion induced by different drugs and concentrations during extended culture periods, challenges arise in conducting measurements on a large scale and managing variability in drug and culture conditions. A slightly large standard deviation was observed in the outcomes of the sensors (FIGS. 5C-5E). This result could be attributed to potential variations from a combination of steps during the sampling and sensing processes. The observed trend differences might likely arise from inherent variability among sensors (though minimal variation was seen in calibration steps), the small sample size of cuboids, and potential variations in baseline viability. Nevertheless, despite these intrinsic variations, the sensor platform effectively captured the CytC production trends associated with different drug treatments. The result underlines the system's potential as a promising platform for enhancing the accuracy of ex vivo anti-cancer drug screening on micro dissected biopsies.

The disclosed platforms can be implemented in the context of long-term monitoring of CytC in culture using electrochemical aptasensors with slow k_{off} rates, including with any of various enhancements or other considerations. Factors like sensor saturation points and the need for sensor regeneration, especially after calibration, for a slow dissociation rate can be developed. For instance, altering the aptamer sequence for lower binding affinity or modifying the sensor surface to amplify the signal response can be implemented to refine the platform's performance. This strategy can be used not only for CytC but also for the development of aptamers targeting other relevant cancer biomarkers.

The disclosed sensing platform is an encouraging step in the real-time detection of CytC in long-term cultures of intact tumor biopsies. Other experiments can further validate this approach. These can include 1) the utilization of various biopsies from diverse patients; 2) the establishment of extended culture periods to enable cross-comparisons among multiple biomarkers, facilitating the accumulation of meaningful data; and 3) the comparison of these results with alternative approaches in patient studies. Nevertheless, the versatility of the presented technology allows for its adaptation to the multiplexing of a wide array of relevant biomarkers from human tissues and patient-derived samples, further advancing the real-time monitoring of cancer disease models and cancer drug screening platforms. The platform's scalability enables its future adaptation to multiplexed monitoring of diverse biomarkers, offers comprehensive insight into cellular responses, and enhances its applicability within intricate culture environments. These advancements contribute to a more in-depth understanding of intact biopsy responses to combination therapies, thus expediting the development and implementation of enhanced treatment modalities for cancer.

### Materials and Methods

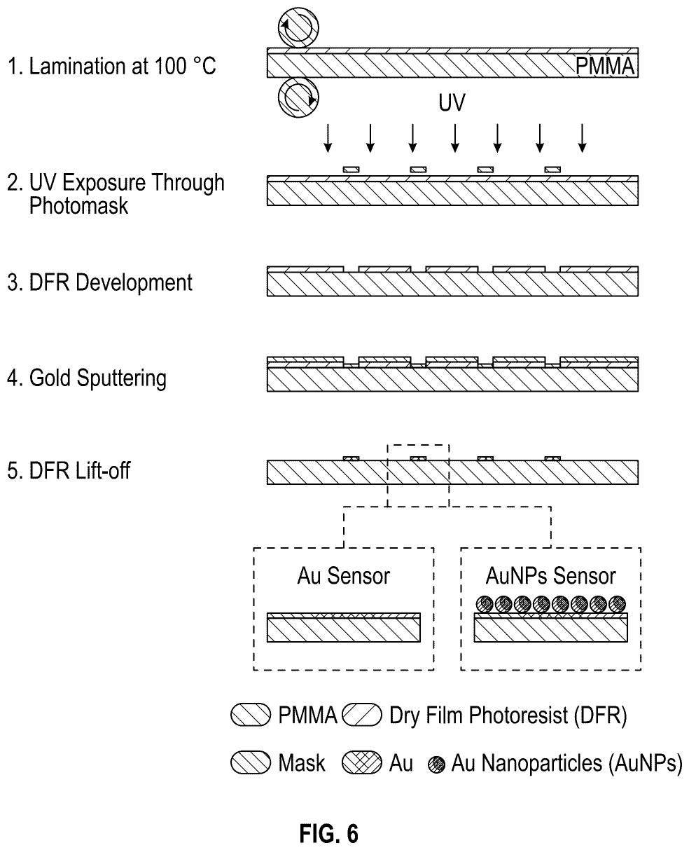

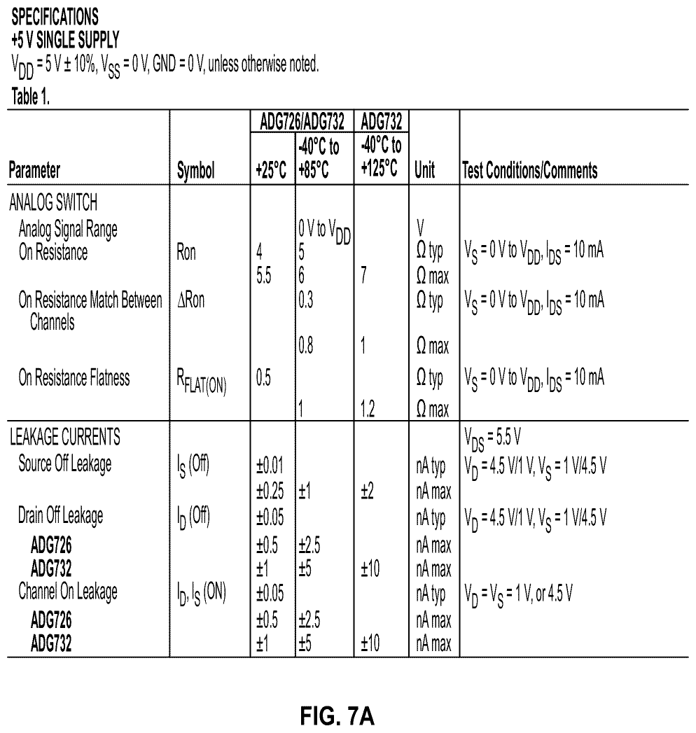

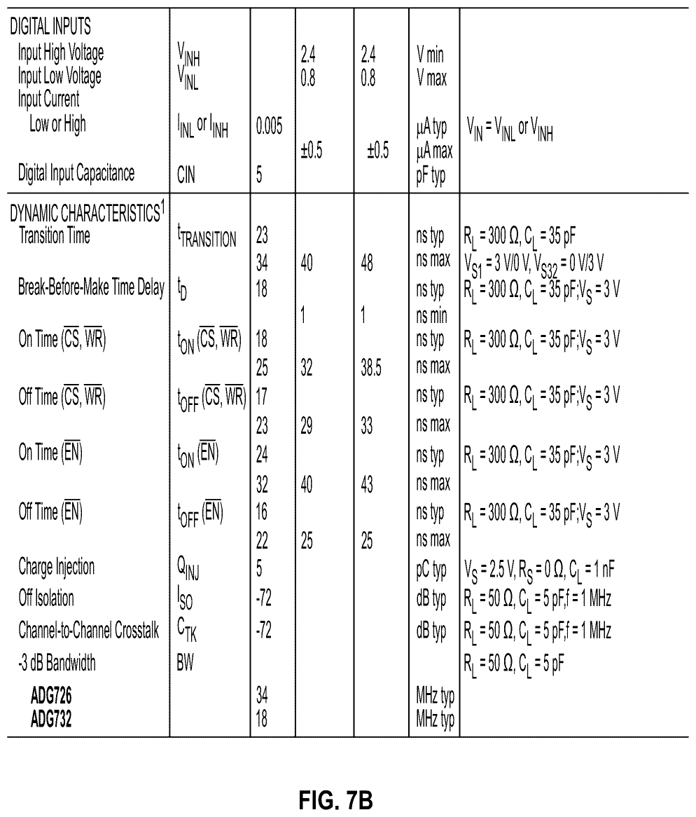

Regents and materials. All reagents were purchased from Thermo Fisher Scientific (MA, USA) unless stated otherwise. The recombinant human cytochrome c was purchased from RayBiotech Life, Inc (GA, USA). Cytochrome C aptamer (100 mM) and IDTE pH 7.5 (1×TE solution) were purchased from Integrated DNA Technologies Inc. (IA, USA). Methylene blue succinimidyl ester (MB-NHS) was purchased from Biotium (CA, USA). 6-Mercapto-1-hexanol (MCH) was purchased from TCI America Inc. (OR, USA). UltraPure DNase/RNase-free distilled water was purchased from Thermo Fisher Scientific (MA, USA). Phosphate-buffered saline powder (PBS; 10×, pH 7.4), Urea powder (Bioreagent), Sodium Chloride (ACS reagent), Nafion perifluorinated resin solution (5 wt. %), Gold (III) chloride hydrate (HAuCl₄·xH₂O, ˜50% Au basis) were purchased from Sigma Aldrich (MO, USA). PMMA was purchased from Astra Products, Inc. (NY, USA). Dry film photoresist (DF-1050) was generously given by Nagase ChemteX (OH, USA). Ag/AgCl (3M KCl) was purchased from BASi (IN, USA). Deionized water (DI) was purified using Milli-Q (Millipore, Bedford, MA). Drugs were diluted from DMSO stocks (10-200 mM), purchased from MedChem Express, and cisplatin (MedChemExpress, 3 M stock in DI water). Fabrication of microfluidic chip and integration of sensors on the platform. AutoCAD 2021 (Autodesk, CA, USA) was used to design the sensor pattern and the patterns for the sealing layer, connectors, insulation layer, loading frame, and a bottomless 24-well plate. Employing CO₂ laser micromachining on PMMA, all layers were fabricated with different optimal laser powers and speeds. The CO₂ laser system used (VLS3.60, Scottsdale, USA) has a wavelength of 10.6 μm and a maximum power of 30 W. Example depictions of sensor fabrication and form factors are shown at FIGS. 6 and 12A-12C. To fabricate the electrode (FIG. 1C), a high-resolution photomask (10,160 DPI resolution, Fine Line Imaging, CO, USA) of the sensor pattern was generated. Subsequently, dry film photoresist (Nagase ChemteX, OH, USA) was used as a sacrificial layer for Au deposition. This process was initiated by hot rolling the negative dry film photoresist (50 μm) onto the PMMA, employing a double-side hot roll laminator (SKY-335R6, Sky-Dsb Co., Seoul, KR). Afterward, the electrode pattern was affixed onto the dry film resist layer using a contact mask aligner (2001, Myriad Semiconductor, AR, USA). It was exposed to UV light with exposure energy ranging from 250-300 mJ/cm² for 30 s. Following this, a four-step development procedure was followed: 1) Applying fresh running cyclohexanone through three cycles of 60 seconds each, 2) subjecting the samples to sonication in cyclohexanone for 1 min, 3) washing them with isopropanol for at least 1 min., 4) rinsing them under running DI water. Subsequently, the samples were dried using an air gun and stored them in an airtight container before depositing Au using electron beam evaporation. To retain the option of removing the sacrificial layer post-Au deposition, one can choose to not subject the photoresist layer to the final hard bake step. The next step involved treating the samples with oxygen plasma utilizing a reactive ion etching system (100 W, 75 mT, 80 seem 02, 1 min). Subsequently, a Cr/Au (10/100 nm) layer was deposited through electron beam evaporation, after which the gold planar electrode array was formed by physically lifting the dry film resist sacrificial layer. 3M™ High-Strength Acrylic Adhesive 300LSE (MN, USA) was manually applied to line the insulation layer (PMMA 0.5 mm thick), loading frame, and well layer (PMMA 6.4 mm thick, 1227T569, McMaster-Carr, Elmhurst, IL) before laser cutting (FIG. 1B). Next, the sensor layers were assembled along with the insulation layer and loading frame by removing the 3M300LSE liner and placing them under a heat press (Model 3912, Carver, IN, USA) at 200 psi for 5 minutes at room temperature. After this assembly, the sensors were subjected to cleaning and Ag/AgCl deposition steps. The same procedure as mentioned above was followed to assemble the well layer. However, this assembly step was only performed before the sensor was ready to be incubated with the CytC aptamer. Cleaning process for WE and RE surfaces. A gold cleaning protocol was performed, employing a combination of H₂O₂+KOH solution and KOH sweeping to clean the Au surface. For this purpose, a solution of 50 mM KOH and 25% H₂O₂ was applied to bare Au surfaces (including all surfaces intended for WE, RE, and CE) for 5 min (extending the duration could potentially lead to lifting off of the Au surface). Subsequently, the surfaces were thoroughly rinsed with DI water. Following the initial treatment, each electrode was subjected to a potential sweep ranging from 0.2 to 1.2 mV (vs. Ag/AgCl) at a scan rate of 50 mV/s in a solution of 50 nM KOH. Afterward, the electrodes were rinsed with DI water. A standard glass-bodied reference electrode with a porous junction (Ag/AgCl with 3M KCl) and a Pt wire were employed as the reference and counter electrodes, respectively. The AuNPs electrodeposition process was monitored and documented.

Fabrication and characterization of Ag/AgCl film for the reference electrode. Once the Au surface had been cleaned, silver was electrodeposited onto the surface at −0.3 V for 300 seconds using a plating solution containing 250 mM silver nitrate, 750 mM sodium thiosulfate, and 500 mM sodium bisulfite. Subsequently, the electrode underwent treatment with 10% hydrochloric acid (HCl) for 1 minute. An aqueous solution comprising 0.1 M potassium chloride (KCl) and 0.01 M HCl was employed for anodic chlorination. Linear sweep voltammetry (LSV) was conducted, spanning the open circuit potential (OCP) to 0.4 V at a scan rate of 20 mV/s. This step was followed by ten cyclic voltammetry (CV) cycles within the 0.1 to 0.3 V range versus Ag/AgCl, employing a fixed scan rate of 100 mV/s. Subsequently, the electrode was thoroughly rinsed with deionized (DI) water and then incubated with 10 μL of 3.5 M KCl for 1 minute. The KCl droplet was subsequently removed, and the electrode was once again rinsed extensively with DI water. After drying the electrode with nitrogen (N₂) gas, it was coated with a 10 μL droplet of 0.5% Nafion solution and left to dry overnight. A standard glass-bodied reference electrode with a porous junction (Ag/AgCl with 3M KCl) and a Pt wire were utilized as the reference and counter electrode, respectively.

Modify sensor platform to become injection analysis platform for characterization of binding kinetics. The existing platform was modified by introducing an additional PMMA layer to establish channels and the well layer altered to create a sealed chamber for flow injection. The PMMA layer (0.3 mm thick) was lined with 3M300LSE adhesive before being precisely cut using a CO₂ laser. The culture medium was injected through tubing (Tygona, Cole-Parmer, IL, USA) connected to the injector loop at a 2.5 mL/min flow rate using a Fusion 200 syringe pump (Chemyx, Inc, TX, USA). Following a 5-minute equilibration period during which the sensors were exposed to continuous medium flow, CytC was introduced into the flow as the target compound.

Cyt C aptamer sequences. The Cyt C aptamer sequence is 5′-/ThioMC6-D/CC GTG TCT GGG GCC GAC CGG CGC ATT GGG TAC GTT GTT GC/AmMO/-3′ (SEQ ID NO:1). Specifically, the DNA oligo was modified with a disulfide (S—S) bond at the 5′ terminus through a six-carbon (C6) spacer and an amino modifier at 3′ terminus. It was HPLC purified and arrived as lab ready (100 mM in IDTE, pH 8.0) and stored at −20° C. until used. The redox reporters MB-NHS were conjugated to the 3′-terminus of amino-modified CytC aptamer through succinimide ester coupling following previously reported protocols. In short, MB-NHS (5 mg) was first dissolved in DMSO to a final concentration of 10 mg/ml and stored as aliquots in the dark at −20° C. MB-NHS (10 mg/mL) was added with 100 mM aptamer solution so that NHS dye solution was 50 molar excess dye to aptamer ratio. The mixture was vortexed for 10 s to help disperse and allowed to react for 4 hrs at room temperature in the dark.

Functionalization of aptamer on the working electrode. The working electrode was rinsed with copious deionized water (DI) and dried under N₂ gas before aptamer immobilization. TCPE stock (100 mM) was prepared with UltraPure DNase/RNase-free distilled water that remained fresh for 3 months when stored in the dark at −20° C. Conjugated MB-CytC aptamers were reduced by 100 mM TCPE with final concentration 1,000-fold of aptamer concentration at room temperature for 1 hour to cleave the S—S bond. Consecutively, the oligos were then diluted to 5 mM using TE buffer and vortexed for 10 s to help disperse. Five microliters of CytC aptamer (5 mM) solution were dropped on the working electrode and incubated in an airtight humidity petri dish at 4° C. overnight (˜15 hrs). The aptamer-immobilized electrodes were subsequently rinsed with copious DI water. MCH was diluted to 10 mM with UltraPure DNase/RNase-free distilled water before dropping onto each working electrode (5 mL) and incubated airtight at room temperature for at least 7 hrs. Lastly, the sensors were rinsed with copious DI water and stored in filter PBS (1×, pH 7.4) until used.

Electrochemical signal measurement. All the electrochemical measurements were performed using a Digi-Ivy potentiostat (DY-2219, Digi-Ivy, Austin, TX, USA). For the SWV measurements, the sensors were integrated from 0.0 to −0.5 V (versus Ag/AgCl) with 40-mV amplitude and a voltage increment of 1 mV suitable frequency setting (ranging from 10 Hz to 1,000 Hz). A conventional three-electrode cell was used in electrodeposition, Ag/AgCl with 3M KCl as reference electrode, and platinum wire (0.05 mm diameter, Alfa Aesar, MA, USA) as counter electrode.

Quantitative analysis and characterization of the Cyt C aptamer sensor. The CytC sensors were characterized in cultured medium DMEM-F12-10% FBS with spiked human CytC recombinant (reconstituted in PBS). The SWV measurement for aptasensors was scanned from −0.5 V to 0 V with 40-mV amplitude and a step potential of 1 mV. The frequency is 50 Hz and 1,000 Hz.

Response of sensor for regeneration. Due to the slow k_{off} rate, the disclosed sensor platform includes regeneration after calibration before in vitro and ex vivo testing. Various regeneration methods were evaluated to identify one suitable for the platform. Multiple chemical regenerations were applied with different pH, such as sodium hydroxide solution (0.05 M, pH 10), glycine in sodium hydroxide buffer (0.1 M, pH 8), and glycine in hydrochloric acid (0.1 M, pH 3.0). Additionally, a high-concentration sodium chloride solution (2M) was used to adjust ionic strength. High-concentration urea (6M, pH 7.0) was employed for regeneration, and thermal regeneration using PBS at 60° C. was also considered. As a result, although the signal gain differed slightly from the original due to the removal of some loosely bound aptamers, the regeneration with urea (6M) yielded favorable outcomes without compromising sensor functionality.

Hardware design. The primary elements include the analog switches and multiplexer, the RFduino ESP32 microcontroller, and the PCB circuitry to interface with the sensor platform and multiplexing chips. Eagle 2022 (Autodesk, USA) software was used to design the circuit schematics. Subsequently, the component layout and wire connections for the two-layer PCB prototype were created, with one dedicated to interfacing with the sensor platform and another for housing the multiplexing chips. Both designs were fabricated and partially assembled using a PCB manufacturing service (JLCPCB, HK). The sensor platform was connected to the PCB using standard pin connectors and a connection established between the PCB and the multiplexing housing board using FFC.

Construction of multiplexing system. For the example implementation, the RFduino ESP32 microcontroller (HiLetgo) was used, which features a low-cost 32-bit LX6 microprocessor. The ESP32 chip package includes GPIO ports and an I2C bus for interfacing with peripheral components. Moreover, the ESP32 is compatible with the Arduino development environment. In order to achieve multichannel operations, multiplexer chips were employed. A multiplexer is a device that features multiple inputs, a single output, and control signals to determine which input is routed to the output. For this specific application, three analog-switched 32-to-1 multiplexer components (Analog Devices Inc., ADG732) were used. These components have on-chip latches that facilitate the necessary hardware logic, allowing for control through GPIO. All WE, RE, and CE of the sensor array were connected to the corresponding WE, RE, or CE multiplexer chip. The multiplexer chips were connected in parallel to the ESP32's+5V and GND pins for their power supply. GPIO pins on the ESP32 were connected to the 5 control pins on the multiplexer chips. These pins were configured to output either a +5V or 0V signal, corresponding to a 1 or a 0. The multiplexers then outputted a signal that depended on the configuration of the 5 input signals (ADG732 Datasheet, Analog Devices). The setup allows for quick and effortless shifting between different sensors on the array.

Cell culture and drug screening. U-87 MG cells (ATTC) were grown in DMEM-F12 supplemented with 10% FBS and penicillin/streptomycin (Invitrogen). Cell passages were performed biweekly. Mouse tumor models and microdissection cuboid procedure. Mice were managed following institutional guidelines and under protocols endorsed by the Animal Care and Use Committee at the University of Washington (Seattle, USA). To create xenograft tumor models, male athymic nude mice (Jackson Laboratories, Foxn1nu) aged 6-10 weeks received subcutaneous injections in the flank region (1-2 million cells in 200 μL of serum and antibiotic-free DMEM-F12 medium). The mice were euthanized before the tumor volume exceeded 2 cm² (within 2-4 weeks). The cuboidal-shaped micro dissected tissues were processed using a previous protocol. In brief, the tissue was first cut into 400 μm-thick slices using a 5100 mz vibratome (Lafayette, Instrument) in an ice-cold HBSS solution (1×) the slices transferred into ice-cold DMEM-F12 with 15 mM HEPES (Invitrogen). The slices were cut into cuboids using a standard Mcllwain tissue chopper (Ted Pella, Inc., USA) with two sets of orthogonal cuts separated by rotation of the sample holder. After separating the cuboids in DMEM-F12-HEPES by gently pipetting them up and down, they were filtered through a 750 μm and 300 μm filters (Pluriselect, USA). The cuboids were washed three times with DPBS, followed by a final fresh wash with DMEM-F12 containing HEPES. Subsequently, the cuboids were cultured in DMEM-F12-10% FBS overnight, allowing them to recover and remove any cell damage from handling before seeding them onto the sensors.

Human colorectal cancer tumor supernatant. Colorectal tumor biopsy was obtained from a 43-year-old male with metastatic cancer who had been off treatment for a year. The cuboidal shaped DT tissues were processed using a previously established protocol as stated above. Subsequently, the cuboids were cultured under different drug conditions: FOLFOX (1 μg/mL 5FU+1 μg/mL Oxaliplatin), FOLFIRI (1 μg/mL 5FU+2 g/mL Irinotecan), Staurosporine (1 μM), and a vehicle control (0.2% DMSO) for three days in modified William's E Media. Electrochemical recording from the supernatant of micro dissected tumor tissue culture. The sensor platform was calibrated using human CytC recombinant ranging from 0-10,000 ng/mL. Following the calibration, the sensors were regenerated with 6 M urea for 5 min and stored in PBS at 4° C. for next-day measurement. First, SWV measurements were conducted to collect the baseline signal, utilizing a potential range of −0.5 V to 0 V, with an amplitude of 4 mV and pulse frequencies set at 50 and 1,000 Hz. An appropriate volume of supernatant samples was added to achieve final concentrations that were 10-fold diluted compared to the stock solution.

Encapsulation of micro dissected tumor tissues using Cellink bioink. After the overnight recovery, the cuboids were transferred into a 96-well plate with 4 cuboids in each well to prepare for their seeding onto sensors. Cuboids were transferred using a pipette with wide bore tips made by cutting off the ends. Cellink bioink, diluted to a 50% concentration with DPBS, was mixed well in a 1 mL Eppendorf tube. After removing the medium from the 96-well plate, 10 μL of the diluted Cellink bioink was added to each well and immediately transferred the encapsulated cuboids in bioink onto the WE surface. To crosslink the gel around the encapsulated tissues and ensure its mechanical stability on the WE, 150 μL of a 50 mM CaCl₂ crosslinking agent (CELLINK, CA, USA) was introduced for 10 min. After three rounds of washing with DPBS, a growth medium was added, DMEM-F12-10% FBS, for subsequent culture.

Ex vivo electrochemical recording from microdissected tumor tissues cultured with drugs. The sensor platform was calibrated using human CytC recombinant ranging from 0-25000 ng/mL in DMEM-F12-10% one day before the incubation. After calibration, the sensors were regenerated with 6 M urea for 5 min and stored in PBS at 4° C. for next-day incubation. The encapsulated cuboid sensor platform was placed in a 37° C. incubator for a minimum of 4 hrs to establish the baseline stability. Subsequently, SWV measurements were performed to collect the baseline signal, utilizing a potential range of −0.5 V to 0 V, with an amplitude of 4 mV and pulse frequencies set at 50 and 1,000 Hz. Drugs were diluted in the cell culture medium for the drug treatment experiment and then transferred to the designated wells. The sensor platform was cultured in the 37° C. incubator during the experiment and removed for measurement every 12 hrs over 48 hrs. Tissue processing and staining, brightfield microscopy, and image analysis. Live cuboids were stained for 1 hr at 37° C. using SYTOX green (SG; Invitrogen, 0.01 μM) with or without Hoechst (H; Invitrogen, 16 μM). Epifluorescence and brightfield microscopy of the cuboids were performed using a BZ-X800 microscope (Keyence, IL, USA) at 2× magnification. FIJI software was used to analyze SG intensity as follows. First, the background fluorescence determined from empty areas was subtracted. Next, cuboid regions were established from the Hoechst channels using thresholding and watershed techniques on a binary image. With the Analyze Particles function, the mean SG fluorescence was acquired, which was subsequently normalized to the average value of untreated cuboids.

Electrochemical data analysis. The frequency of the applied square wave influences the signaling of the aptasensor. Square wave voltammetry can be adjusted to result in either an increase (Signal-ON) or a decrease (Signal-OFF) in peak current upon the introduction of the target molecule (FIG. 2E). To address signal drift and improve signal strength during ex vivo measurements, voltammograms can be captured at two different frequencies, and these then transformed into “Kinetic Differential Measurement” (KDM) values. These KDM values are calculated by subtracting the normalized peak currents observed at the Signal-ON and Signal-OFF frequencies and dividing them by their average. To create a calibration curve, the averaged KDM values obtained in vitro across a range of target concentrations are fitted to a Hill-Langmuir isotherm equation (Eq. 1). The calibration curve fitting enables the extraction of all the parameters nH, K1/2, KDMmin, and KDMmax.

$$K D M = K {DM}_{min} + \frac{( K D {M}_{max} - K D {M}_{min} ) * {[ {C}_{CytC} ]}^{n H}}{{[ {C}_{CytC} ]}^{n H} + {K}_{1 / 2}^{n H}} \qquad Eq . 1$$

nH represents the Hill coefficient, which measures the degree of binding cooperativity.
K_{1/2} denotes the midpoint of the binding curve.
KDM refers to the observed KDM value at the given target concentration.
KDMₘᵢₙ represents the KDM value recorded in the absence of the target.
KDMₘₐₓ represents the anticipated KDM value when the target is fully saturated.
CCytc refers to the concentration of the analyte of interest (e.g., CytC).

Once derived from a calibration curve, these parameters facilitate the conversion of aptasensor output into estimations of the target concentration (Eq. 2).

$$[ {C}_{CytC} ] = \frac{{K}_{1 / 2}^{n H}}{{( \frac{K D {M}_{max} - K D {M}_{min}}{K D M - K D {M}_{min}} - 1 )}^{1 / n H}} \qquad Eq . 2$$

Statistical analyses. GraphPad Prism 10 (Boston, MA, USA) was used to perform statistical tests and data visualization. MATLAB (R2021a, USA) was used to analyze all experiment data. First, the raw data was exported to MATLAB, then the MB redox peak currents extracted from record voltammograms, and KDM values calculated. Subsequently, the MB redox peak currents were extracted from recorded voltammograms to compute KDM values. Titration concentration was entered and average KDM values into Eq. 1 in MATLAB to fit the data to a Hill-Langmuir isotherm. The data was then fitted to Eq. 1 with the following constraints: −∞<KDMₘₐₓ<∞, 1×e⁻¹²<K_{1/2}<1, −∞<KDMₘᵢₙ<∞, 0<nH<10.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. An integrated electrochemical aptamer-based (E-AB) sensor configured to measure a concentration of an analyte, the integrated E-AB sensor comprising: an aptamer operably connected to a conductive substrate; circuitry configured to conduct square wave voltammetry (SWV) to apply an electric current to the conductive substrate; and circuitry configured to conduct kinetic differential measurement (KDM) to determine concentration of the analyte based on changes in the electric current caused by conformational changes in the aptamer upon an interaction between the aptamer and the analyte.

Embodiment 2. The integrated E-AB sensor of Embodiment 1, wherein the aptamer comprises: a redox reporter configured to modify an electric current to and/or from the conductive substrate of the integrated E-AB sensor; and a nucleic acid aptamer comprising a polynucleotide sequence configured to interact with the analyte, wherein an interaction between the polynucleotide sequence and the analyte causes a conformational change in the nucleic acid aptamer, a positional change in the redox reporter, and a change in the electric current of the integrated E-AB sensor; wherein the electric current is applied with circuitry configured to conduct SWV and the change in the electric current is processed with circuitry configured to conduct KDM to determine concentration of the analyte.

Embodiment 3. The integrated E-AB sensor of any one of Embodiments 1-2 or any other Embodiment, wherein a form factor of the integrated E-AB sensor comprises a dip-stick form factor.

Embodiment 4. The integrated E-AB sensor of any one of Embodiments 1-3 or any other Embodiment, wherein the nucleic acid aptamer comprises deoxyribonucleic acid (DNA) and the polynucleotide sequence comprises a DNA sequence.

Embodiment 5. The integrated E-AB sensor of any one of Embodiments 1-4 or any other Embodiment, wherein the redox reporter comprises methylene blue (MB).

Embodiment 6. The integrated E-AB sensor of any one of Embodiments 1-5 or any other Embodiment, wherein the redox reporter is linked to the nucleic acid aptamer at a terminus of the nucleic acid aptamer.

Embodiment 7. The integrated E-AB sensor of any one of Embodiments 1-6 or any other Embodiment, wherein the terminus is the 3′ terminus of the nucleic acid aptamer.

Embodiment 8. The integrated E-AB sensor of any one of Embodiments 1-7 or any other Embodiment, wherein the analyte comprises Cytochrome C (Cyt-C) and the polynucleotide sequence comprises a DNA sequence specific for interaction with Cyt-C.

Embodiment 9. The integrated E-AB sensor of any one of Embodiments 1-8 or any other Embodiment, wherein the analyte comprises Cyt-C and the polynucleotide sequence comprises a DNA sequence specific for interaction with Cyt-C, wherein the DNA sequence specific for interaction with Cyt-C comprises a DNA sequence that is at least 80% identical to SEQ ID NO:1.

Embodiment 10. The integrated E-AB sensor of any one of Embodiments 1-9 or any other Embodiment, wherein the analyte comprises Cyt-C and the polynucleotide sequence comprises a DNA sequence specific for interaction with Cyt-C, wherein the DNA sequence specific for interaction with Cyt-C is SEQ ID NO:1.

Embodiment 11. The integrated E-AB sensor of any one of Embodiments 1-10 or any other Embodiment, wherein the circuitry configured to conduct SWV uses a combined square wave applied to the conductive substrate of the integrated E-AB sensor.

Embodiment 12. The integrated E-AB sensor of any one of Embodiments 1-11 or any other Embodiment, wherein the circuitry configured to conduct KDM to determine concentration of the analyte is configured to calculate concentration of the analyte according to:

$$[ Analyte ] = \frac{K D {M}_{min}}{{( \frac{K D {M}_{max} - K D {M}_{min}}{K D M - K D {M}_{min}} - 1 )}^{n H}}$$

wherein:
[Analyte] is the concentration of the analyte, optionally in ng/mL;
KDMₘᵢₙ is KDM observed in absence of the analyte;
KDMₘₐₓ is KDM expected at saturation of the analyte; and
nH is Hill coefficient.

Embodiment 13. The integrated E-AB sensor of any one of Embodiments 1-12 or any other Embodiment, wherein the circuitry configured to conduct SWV and the circuitry configured to conduct KDM are implemented as a non-transitory computer-readable storage medium having instructions stored thereon which, when executed by the processor, configure the processor to: apply the electric current according to SWV; detect the change in the electric current; and process the change in the electric current according to KDM.

Embodiment 14. A method for measuring an analyte with an integrated E-AB sensor, the method comprising: contacting a conductive substrate of the integrated E-AB sensor with a sample comprising an analyte; applying, with circuitry configured to conduct SWV, electric current to the conductive substrate; and processing, with circuitry configured to conduct KDM, a change in an electric current associated with an interaction between an aptamer of the integrated E-AB sensor and the analyte.

Embodiment 15. The method of Embodiment 14, wherein the integrated E-AB sensor comprises: the aptamer operably connected to the conductive substrate; circuitry configured to conduct SWV to apply the electric current to the conductive substrate; and circuitry configured to conduct KDM to determine concentration of the analyte based on changes in the electric current caused by conformational changes in the aptamer upon an interaction between the aptamer and the analyte.

Embodiment 16. The method of any one of Embodiments 14-15 or any other Embodiment, wherein the sample comprises a tissue sample within a solution and the analyte is from, or is produced by, the tissue sample.

Embodiment 17. The method of any one of Embodiments 14-16 or any other Embodiment, wherein the tissue sample is selected from the group consisting of: a cuboid tissue sample, a tissue slice, an organoid tissue, and any combination thereof.

Embodiment 18. The method of any one of Embodiments 14-17 or any other Embodiment, wherein the analyte corresponds with a response of a cuboid tissue sample of the sample to a treatment.

Embodiment 19. A kit for measurement of an analyte, the kit comprising: an integrated E-AB sensor, comprising: an aptamer operably connected to a conductive substrate of the E-AB sensor; circuitry configured to conduct SWV to apply an electric current to the conductive substrate; and circuitry configured to conduct KDM to determine concentration of the analyte based on a change in an electric current caused by a conformational change in the aptamer upon an interaction between the aptamer and the analyte; and instructions for a use of the kit in a method for measuring the analyte with the kit.

Embodiment 20. The kit of Embodiment 19, wherein the integrated E-AB sensor is according to any one of Embodiments 1-13 and/or comprises: the aptamer is according to any one of Embodiments 1-13 and/or is operably connected to the conductive substrate; circuitry configured to conduct SWV to apply the electric current to the conductive substrate; and circuitry configured to conduct KDM to determine concentration of the analyte based on changes in the electric current caused by conformational changes in the aptamer upon an interaction between the aptamer and the analyte.

While illustrative embodiments have been illustrated and described, it will be appreciated that various changes can be made therein without departing from the spirit and scope of the invention.

The embodiments of the invention in which an exclusive property or privilege is claimed are defined as follows:

1. An integrated electrochemical aptamer-based (E-AB) sensor configured to measure a concentration of an analyte, the integrated E-AB sensor comprising:
an aptamer operably connected to a conductive substrate;
circuitry configured to conduct square wave voltammetry (SWV) to apply an electric current to the conductive substrate; and
circuitry configured to conduct kinetic differential measurement (KDM) to determine concentration of the analyte based on changes in the electric current caused by conformational changes in the aptamer upon an interaction between the aptamer and the analyte.

2. The integrated E-AB sensor of claim 1, wherein the aptamer comprises:
a redox reporter configured to modify an electric current to and/or from the conductive substrate of the integrated E-AB sensor; and
a nucleic acid aptamer comprising a polynucleotide sequence configured to interact with the analyte, wherein an interaction between the polynucleotide sequence and the analyte causes a conformational change in the nucleic acid aptamer, a positional change in the redox reporter, and a change in the electric current of the integrated E-AB sensor;
wherein the electric current is applied with circuitry configured to conduct SWV and the change in the electric current is processed with circuitry configured to conduct KDM to determine concentration of the analyte.

3. The integrated E-AB sensor of claim 1, wherein a form factor of the integrated E-AB sensor comprises a dip-stick form factor.

4. The integrated E-AB sensor of claim 2, wherein the nucleic acid aptamer comprises deoxyribonucleic acid (DNA) and the polynucleotide sequence comprises a DNA sequence.

5. The integrated E-AB sensor of claim 2, wherein the redox reporter comprises methylene blue (MB).

6. The integrated E-AB sensor of claim 2, wherein the redox reporter is linked to the nucleic acid aptamer at a terminus of the nucleic acid aptamer.

7. The integrated E-AB sensor of claim 6, wherein the terminus is the 3′ terminus of the nucleic acid aptamer.

8. The integrated E-AB sensor of claim 2, wherein the analyte comprises Cytochrome C (Cyt-C) and the polynucleotide sequence comprises a DNA sequence specific for interaction with Cyt-C.

9. The integrated E-AB sensor of claim 2, wherein the analyte comprises Cyt-C and the polynucleotide sequence comprises a DNA sequence specific for interaction with Cyt-C, wherein the DNA sequence specific for interaction with Cyt-C comprises a DNA sequence that is at least 80% identical to SEQ ID NO:1.

10. The integrated E-AB sensor of claim 2, wherein the analyte comprises Cyt-C and the polynucleotide sequence comprises a DNA sequence specific for interaction with Cyt-C, wherein the DNA sequence specific for interaction with Cyt-C is SEQ ID NO:1.

11. The integrated E-AB sensor of claim 1, wherein the circuitry configured to conduct SWV uses a combined square wave applied to the conductive substrate of the integrated E-AB sensor.

12. The integrated E-AB sensor of claim 1, wherein the circuitry configured to conduct KDM to determine concentration of the analyte is configured to calculate concentration of the analyte according to:
$$[ Analyte ] = \frac{K D {M}_{min}}{{( \frac{K D {M}_{max} - K D {M}_{min}}{K D M - K D {M}_{min}} - 1 )}^{n H}}$$
wherein:
[Analyte] is the concentration of the analyte, optionally in ng/mL;
KDMₘᵢₙ is KDM observed in absence of the analyte;
KDMₘₐₓ is KDM expected at saturation of the analyte; and
nH is Hill coefficient.

13. The integrated E-AB sensor of claim 1, wherein the circuitry configured to conduct SWV and the circuitry configured to conduct KDM are implemented as a non-transitory computer-readable storage medium having instructions stored thereon which, when executed by the processor, configure the processor to:
apply the electric current according to SWV;
detect the change in the electric current; and
process the change in the electric current according to KDM.

14. A method for measuring an analyte with an integrated E-AB sensor, the method comprising:
contacting a conductive substrate of the integrated E-AB sensor with a sample comprising an analyte;
applying, with circuitry configured to conduct SWV, electric current to the conductive substrate; and
processing, with circuitry configured to conduct KDM, a change in an electric current associated with an interaction between an aptamer of the integrated E-AB sensor and the analyte.

15. The method of claim 14, wherein the integrated E-AB sensor comprises:
the aptamer operably connected to the conductive substrate;
circuitry configured to conduct SWV to apply the electric current to the conductive substrate; and
circuitry configured to conduct KDM to determine concentration of the analyte based on changes in the electric current caused by conformational changes in the aptamer upon an interaction between the aptamer and the analyte.

16. The method of claim 14, wherein the sample comprises a tissue sample within a solution and the analyte is from, or is produced by, the tissue sample.

17. The method of claim 16, wherein the tissue sample is selected from the group consisting of: a cuboid tissue sample, a tissue slice, an organoid tissue, and any combination thereof.

18. The method of claim 17, wherein the analyte corresponds with a response of a cuboid tissue sample of the sample to a treatment.

19. A kit for measurement of an analyte, the kit comprising:
an integrated E-AB sensor, comprising:
an aptamer operably connected to a conductive substrate of the E-AB sensor;
circuitry configured to conduct SWV to apply an electric current to the conductive substrate; and
circuitry configured to conduct KDM to determine concentration of the analyte based on a change in an electric current caused by a conformational change in the aptamer upon an interaction between the aptamer and the analyte; and
instructions for a use of the kit in a method for measuring the analyte with the kit.

20. The kit of claim 19, wherein the integrated E-AB sensor comprises:
the aptamer operably connected to the conductive substrate;
